(19) United States
(12) Patent Application Publication
(10) Pub. No.: US 2022/0195461 A1
(43) Pub. Date: Jun. 23, 2022

(54) METHODS AND COMPOSITIONS FOR TRANSGENE EXPRESSION

(71) Applicants: University of Iowa Research Foundation, Iowa City, IA (US); Spirovant Sciences, Inc., Philadelphia, PA (US)

(72) Inventors: John F. Engelhardt, Iowa City, IA (US); Ziying Yan, Iowa City, IA (US); Yinghua Tang, Iowa City, IA (US); Eric Yuen, Los Altos, CA (US); Shen Lin, Philadelphia, PA (US)

(21) Appl. No.: 17/603,840

(22) PCT Filed: Apr. 15, 2020
(86) PCT No.: PCT/US2020/028269

§ 371 Date: Oct. 14, 2021

Related U.S. Application Data
(60) Provisional application No. 62/833,979, filed on Apr. 15, 2019.
(60) Provisional application No. 62/926,317, filed on Oct. 25, 2019.
(60) Provisional application No. 62/967,219, filed on Jan. 29, 2020.

(51) Int. Cl. C12N 15/86; A61K 31/69; A61K 31/704; A61K 35/761; A61K 38/05; A61K 38/06; A61K 38/07; A61K 38/17; A61P 11/00

(52) U.S. Cl. CPC C12N 15/86; A61K 31/69; A61K 31/704; A61K 35/761; A61P 11/00; A61K 38/06; A61K 38/07; A61K 38/1709; A61K 38/05

(57) ABSTRACT

The disclosure provides methods of expressing a transgene in a cell, methods of treating disorders in a subject in need thereof, and pharmaceutical compositions. In particular, the methods involve contacting a cell (e.g., a cell of a subject suffering from a disorder such as cystic fibrosis) with a recombinant adeno-associated virus (rAAV) that includes, in one embodiment, an AV.TL65 capsid protein and a polynucleotide that includes a transgene in combination with an augmenter of AAV transduction, thereby expressing the transgene in the cell. The disclosure also provides pharmaceutical compositions that include an rAAV that includes, in one embodiment, an AV.TL65 capsid protein and a polynucleotide including a transgene in combination with one or more augmenters.

### CROSS-REFERENCE TO RELATED APPLICATIONS

This application claims the benefit of the filing date of U.S. application No. 62/833,979, filed on Apr. 15, 2019, U.S. application No. 62/926,317, filed on Oct. 25, 2019, and U.S. application No. 62/967,219, filed on Jan. 29, 2020, the disclosures of which are incorporated by reference herein.

### STATEMENT OF GOVERNMENT RIGHTS

This invention is made with government support under R43HL137583 awarded by the National Institutes of Health. The Government has certain rights in the invention.

### BACKGROUND

Gene therapy using adeno-associated virus (AAV) is an emerging treatment modality, including for treatment of single-gene defects. Cystic fibrosis (CF) is a lethal, autosomal-recessive disorder that affects at least 30,000 people in the U.S. alone, and at least 70,000 people worldwide. The average survival age for CF patients is about 40 years. CF is caused by mutations in the gene encoding the cystic fibrosis transmembrane conductance regulator (CFTR), a channel that conducts chloride and bicarbonate ions across epithelial cell membranes. Impaired CFTR function leads to inflammation of the airways and progressive bronchiectasis. Because of the single-gene etiology of CF and the various CFTR mutations in the patient population, gene therapy potentially provides a universal cure for CF.

Adeno-associated virus (AAV), a member of the human parvovirus family, is a non-pathogenic virus that depends on helper viruses for its replication. For this reason, recombinant AAV (rAAV) vectors are among the most frequently used in gene therapy pre-clinical studies and clinical trials. Indeed, CF lung disease clinical trials with rAAV2 demonstrated both a good safety profile and long persistence of the viral genome in airway tissue (as assessed by biopsy) relative to other gene transfer agents (such as recombinant adenovirus). Nevertheless, gene transfer failed to improve lung function in CF patients because transcription of the rAAV vector-derived CFTR mRNA was not detected.

Therefore, there remains a need in the art for improved methods for transgene expression in AAV-based gene therapy approaches.

### SUMMARY

The disclosure provides, inter alia, methods of expressing a transgene in a cell, methods of treating disorders in a subject in need thereof, and pharmaceutical compositions. In one aspect, the subject is a human neonate. In one aspect, the subject is a human juvenile.

In one aspect, the disclosure features a method of expressing a transgene in a cell, the method comprising contacting the cell with (i) a recombinant adeno-associated virus (rAAV) comprising an AV.TL65 capsid protein, or a variant thereof, and a polynucleotide comprising a transgene; and (ii) an augmenter of AAV transduction, thereby expressing the transgene in the cell. In one embodiment, the variant capsid protein has at least 80% amino acid sequence identity to SEQ ID NO:13.

In some embodiments, the augmenter is a proteasome modulating agent.

In some embodiments, the proteasome modulating agent is an anthracycline, a proteasome inhibitor, a tripeptidyl aldehyde, or a combination thereof.

In some embodiments, the anthracycline is doxorubicin, idarubicin, aclarubicin, daunorubicin, epirubicin, valrubicin, mitoxantrone, or a combination thereof.

In some embodiments, the anthracycline is doxorubicin, idarubicin, or a combination thereof.

In some embodiments, the proteasome inhibitor is bortezomib, carfilzomib, and ixazomib.
In some embodiments, the tripeptidyl aldehyde is N-acetyl-1-leucyl-1-leucyl-1-norleucine (LLnL).
In some embodiments, the cell is contacted sequentially with the rAAV and the augmenter.
In other embodiments, the cell is contacted simultaneously with the rAAV and the augmenter.

In some embodiments, contacting the cell with the rAAV and the augmenter results in an increase in expression of the transgene as compared to contacting the cell with the rAAV alone. In some embodiments, the increase in expression is about 100%, about 200%, about 300%, about 400%, about 500%, about 600%, or greater.

In some embodiments, the contacting comprises administering the rAAV and the augmenter to a subject.

In another aspect, the disclosure features a method of treating a disorder in a subject in need thereof, the method comprising administering to the subject (i) a recombinant adeno-associated virus (rAAV) comprising an AV.TL65 capsid protein and a polynucleotide comprising a therapeutic transgene; and (ii) an augmenter of AAV transduction, wherein the administering results in expression of the transgene in cells of the subject.

In some embodiments, the administering is by inhalation, nebulization, aerosolization, intranasally, intratracheally, intrabronchially, orally, intravenously, subcutaneously, and/or intramuscularly.

In some embodiments, the administering is by inhalation, nebulization, aerosolization, intranasally, intratracheally, and/or intrabronchially.

In some embodiments, the cell is an airway cell. In some embodiments, the cell is an airway epithelial cell. In some embodiments, the airway epithelial cell is a lung epithelial cell.

In some embodiments, the disorder is cystic fibrosis.

In some embodiments, the polynucleotide comprises an F5 enhancer and/or a tg83 promoter. In some embodiments, the F5 enhancer includes the polynucleotide sequence of SEQ ID NO:1 or SEQ ID NO:14, or a variant thereof with at least 80% nucleic acid sequence identity to SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14. In some embodiments, the tg83 promoter includes the polynucleotide sequence of SEQ ID NO:2.

In some embodiments, the transgene is CFTR or a derivative thereof.

In some embodiments, the derivative of CFTR is a CFTRΔR transgene (e.g., a human CFTRΔR transgene). In some embodiments, the human CFTRΔR transgene is encoded by a polynucleotide including the sequence of SEQ ID NO:4, or a variant thereof with at least 80% nucleic acid sequence identity to SEQ ID NO:4.

In some embodiments, the polynucleotide comprises, in a 5′-to-3′ direction, the F5 enhancer, the tg83 promoter, and the CFTRΔR transgene.

In some embodiments, the polynucleotide comprises the sequence of SEQ ID NO:7, or a variant thereof with at least 80% nucleic acid sequence identity to SEQ ID NO:7.

In some embodiments, the polynucleotide further comprises, in the 3′ direction, a 3′ untranslated region (3′-UTR) comprising the sequence of SEQ ID NO:5, or a variant thereof with at least 80% nucleic acid sequence identity to SEQ ID NO:5.

In some embodiments, the polynucleotide further comprises, in the 3′ direction, a synthetic polyadenylation site comprising the sequence of SEQ ID NO:6, or a variant thereof with at least 80% nucleic acid sequence identity to SEQ ID NO:6.

In some embodiments, the polynucleotide further includes a 5′ adeno-associated virus (AAV) inverted terminal repeat (ITR) at the 5′ terminus of the polynucleotide and a 3′ AAV ITR at the 3′ terminus of the polynucleotide. In some embodiments, the 5′ AAV ITR comprises the sequence of SEQ ID NO:15, or a variant thereof with at least 80% nucleic acid sequence identity to SEQ ID NO:15. In some embodiments, the 3′ AAV ITR comprises the sequence of SEQ ID NO:16, or a variant thereof with at least 80% nucleic acid sequence identity to SEQ ID NO:16.

In some embodiments, the polynucleotide comprises: a 5′ AAV ITR comprising the sequence of SEQ ID NO:15, an F5 enhancer comprising the sequence of SEQ ID NO:14 (which may include a 5′ EcoRI site and a 3′ XhoI site, as in SEQ ID NO:1), a tg83 promoter comprising the sequence of SEQ ID NO:2, a 5′ UTR comprising the sequence of SEQ ID NO:3, a hCFTRΔR transgene comprising the sequence of SEQ ID NO:4, a 3′ UTR comprising the sequence of SEQ ID NO:5, a polyadenylation site (s-pA) comprising the sequence of SEQ ID NO:6, and a 3′ AAV ITR comprising the sequence of SEQ ID NO:16.

In some embodiments, the polynucleotide includes the sequence of SEQ ID NO:17, or a variant thereof with at least 80% nucleic acid sequence identity to SEQ ID NO:17.

In some embodiments, the AV.TL65 capsid protein comprises the amino acid sequence of

(SEQ ID NO: 13)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPFGLVEEGAKTAPTGKRIDDHFPKRK
KARTEEDSKPSTSSDAEAGPSGSQQLQIPAQPASSLGADTMSAGGGGPLGD
NNQGADGVGNASGDWHCDSTWMGDRVVTKSTRTWVLPSYNNHQYREIKSGS
VDGSNANAYFGYSTPWGYFDENRFHSHWSPRDWQRLINNYWGFRPRSLRVK
IFNIQVKEVTVQDSTTTIANNLTSTVQVFTDDDYQLPYVVGNGTEGCLPAF
PPQVFTLPQYGYATLNRDNTENPTERSSFFCLEYFPSKMLRTGNNFEFTYN
FEEVPFHSSFAPSQNLFKLANPLVDQYLYRFVSTNNTGGVQFNKNLAGRYA
NTYKNWFPGPMGRTQGWNLGSGVNRASVSAFATTNRMELEGASYQVPPQPN
GMTNNLQGSNTYALENTMIFNSQPANPGTTATYLEGNMLITSESETQPVNR
VAYNVGGQMATNNQSSTTAPTTGTYNLQEIVPGSVWMERDVYLQGPIWAKI
PETGAHFHPSPAMGGFGLKHPPPMMLIKNTPVPGNITSFSDVPVSSFITQY
STGQVTVEMEWELKKENSKRWNPEIQYTNNYNDPQFVDFAPDSTGEYRTTR
PIGTRYLTRPL.

A variant polynucleotide or polypeptide sequence can be at least 80%, at least 85%, at least at least 90%, at least 91%, at least 92%, at least 93%, at least 94%, at least 95%, at least 96%, at least 97%, at least 98%, at least 99%, or more, identical to a native or reference sequence, e.g., a variant polynucleotide of any one of SEQ ID Nos. 1 to 12 and 14 to 17, or a variant polypeptide of SEQ ID NO:13.

In another aspect, the disclosure features a pharmaceutical composition comprising (i) an rAAV comprising an AV.TL65 capsid protein and a polynucleotide comprising a transgene; and (ii) an augmenter of AAV transduction.

In some embodiments, the augmenter is a proteasome modulating agent.

In some embodiments, the proteasome modulating agent is an anthracycline, a proteasome inhibitor, a tripeptidyl aldehyde, or a combination thereof.

In some embodiments, the anthracycline is doxorubicin, idarubicin, aclarubicin, daunorubicin, epirubicin, valrubicin, mitoxantrone, or a combination thereof.

In some embodiments, the anthracycline is doxorubicin, idarubicin, or a combination thereof. In some embodiments, the augmenter is doxorubicin. In other embodiments, the augmenter is idarubicin.

In some embodiments, the proteasome inhibitor is bortezomib, carfilzomib, and ixazomib.

In some embodiments, the tripeptidyl aldehyde is N-acetyl-l-leucyl-l-leucyl-l-norleucine (LLnL).

### BRIEF DESCRIPTION OF THE DRAWINGS

FIGS. 1A and 1B are a series of graphs showing the ratio of luciferase activity in cells treated with AV.TL65+proteasome inhibitor (PI) versus AAV alone (FIG. 1A) and the ratio of LDH activity in cells treated with AV.TL65+PI versus AAV alone (FIG. 1B). These results are from CF (HBE and HTE, dF508/dF508, Passage 0) cells at 4 days after infection (AV.TL65, 10K MOI). Relative Luminescence Units in CF HBE treated with AV.TL65 with PI exceeded 200-fold of AV.TL65 without PI. Toxicity of AV.TL65 with PI as measured by LDH activity was mostly below 150% of AV.TL65 without PI.

FIG. 2 is a series of graphs showing transduction and relative LDH activity for cells of individual donors under the indicated treatment conditions that, when averaged, resulted in the data presented in FIGS. 1A and 1B.

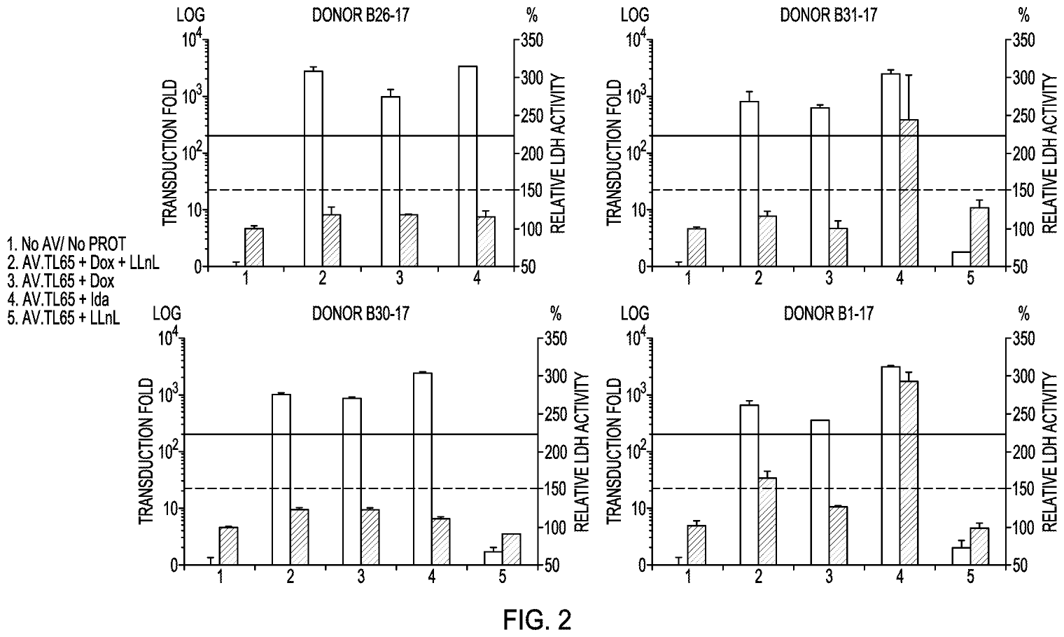

FIGS. 3A-3D. In vitro and in vivo comparison of rAAV vector performance. (A) CF (F508del/F508del) human polarized ALI airway cultures were infected apically with AV1-SP183-hCFTRΔR or the AV.TL65-SP183-hCFTRΔR (MOI=100,000 DRP/cell) in the presence of augmenter. Short circuit current (Isc) measurements were then performed in Ussing chambers at 12-days post-infection. Shown is the ΔIsc response to forskolin/IBMX and GlyH101 (CFTR inhibitor). Data show the mean±SD for n=4 transwells from two donors. Non-infected ALI cultures served as baseline controls (n=4 from two donors). (B) After Isc measurements, two transwell inserts from each group were pooled and lysed to quantify the vector-derived hCFTRΔR mRNA copies by reverse transcriptase quantitative-PCR (RT-qPCR), and normalized to human GAPDH mRNA copies. Values were then expressed as a ratio of hCFTRΔR/GAPDH. Data shows mean±range for n=2. (C) Human and ferret polarized tracheobronchial epithelia at ALI were infected apically with AV.TL65-SP183gLuc at a multiplicity of infection (MOI) of 100,000. DNase-resistant particles (DRP)/cell in the presence of augmenter. Gaussia luciferase activity was measured at 5-days post-infection as relative luminescence units (RLU). Data show the mean±SD for n=6 transwells from two donors of each species. (D) Three-days-old ferrets or one-month-old ferrets were intratracheally infected with AV.TL65-SP183-hCFTRΔR mixed with augmenter (4×10¹⁰ DRP per gram body weight). The mock-infected group was inoculated with PBS with augmenter. The tracheae and lungs were then harvested at 11-days post-infection for quantification of vector-derived hCFTRΔR and endogenous fCFTR mRNA copies by RT-qPCR with GAPDH mRNA copy number normalization. The data represents the ratio (hCFTRΔR/fCFTR) of mRNA copies of hCFTRΔR and fCFTRΔR. Data show the mean+/−SD for n=3 animals in each group. ns, not significantly different.

FIGS. 4A-4C. Repeat dosing of AV.TL65 in neonatal ferrets. (A) Study design involving three groups of neonatal ferrets receiving 0-, 1-, or 2-doses of virus at 1×10¹³ DRP/kg via intra-tracheal administration. The ferrets receiving one dose were administered the reporter vector AV.TL65-SP183-gLuc at 4 wks of age, whereas the ferrets receiving two doses were administered AV.TL65-SP183-fCFTRΔR at 1 wk of age and AV.TL65-SP183-gLuc at 4 wks of age. Plasma and BALF samples were collected at the indicated ages. (B) Gaussia luciferase activity in the plasma at the indicated time points post-delivery of AV.TL65-SP183-gLuc. (C) Gaussia luciferase activity in BALF at 14-days post-delivery of AV.TL65-SP183-gLuc. Results show the mean±SD for n=6 animals per group. The statistical significance was analyzed with one-way ANOVA followed by Tukey's post-test. ns, non-significant. RLU, relative luminescence units.

FIGS. 5A-5C. Repeat dosing of AV.TL65 in juvenile ferrets. (A) Study design involving three groups of juvenile ferrets receiving 0-, 1-, or 2-doses of virus at 1×10¹³ DRP/kg via intra-tracheal administration. The ferrets receiving one dose were administered the reporter vector AV.TL65-SP183-gLuc at 8 wks of age, whereas the ferrets receiving two doses were administered AV.TL65-SP183-fCFTRΔR at 4 wk of age and AV.TL65-SP183-gLuc at 8 wks of age. Plasma and BALF samples were collected at the indicated ages. (B) Gaussia luciferase activity in the plasma at the indicated time points post-delivery of AV.TL65-SP183-gLuc. (C) Gaussia luciferase activity in BALF at 14-days post-delivery of AV.TL65-SP183-gLuc. Results show the mean±SD for n=9-10 animals per group. The statistical significance was analyzed with one-way ANOVA followed by Tukey's post-test: **P<0.01, ****P<0.0001. RLU, relative luminescence units.

FIGS. 6A-6D. Titers of AV.TL65 neutralizing antibodies in the BALF and plasma of infected ferrets. (A, B) Neonatal ferrets samples as collected in FIG. 4A were evaluated for NAbs in the (A) BALF and (B) plasma using transduction inhibition assay. Serial dilutions of BALF or plasma were incubated with AV.TL65-fLuc prior to infection of A549 cells. The titer of NAbs were calculated as the concentration of BALF or plasma (dilution ratio) that resulted 50% inhibition (IC50) of transduction as assessed by firefly luciferase activity. AV.TL65-fLuc only infected cells served as the baseline control and mock-infected cells served as blank. (C, D) Juvenile ferret samples as collected in FIG. 5A were evaluated for NAbs in the (C) BALF and (D) plasma using the above described transduction inhibition assays. Results show the mean±SD for n=6 neonatal animals per group and n=9-10 juvenile animals per group. The statistical significance was analyzed with one-way ANOVA followed by Tukey's post-test: **P<0.01, ****P<0.0001. ns, non-significant.

FIGS. 7A-7B. Development of an ELISA-based assay for quantifying anti-capsid antibody isotypes. Immune plasma was generated from a ferret infected with AV-TL65 to the lung four times at 1-2 months intervals starting at 1 month of age. The naive plasma was derived from a ferret of similar age. ELISA plates were coated with (A) AAV5 or (B) AAV2 and then evaluated for binding of immune and naive ferret plasma. Secondary detection antibodies were against IgG. Results show the mean±range for two technical replicates on each sample.

FIGS. 8A-8F. Quantification of IgG, IgM, and IgA capsid binding antibodies in the plasma of AV.TL65 infected ferrets. (A-F) Quantification of capsid binding antibodies in the plasma of (A-C) neonatal and (D-F) juvenile ferrets for (A,D) IgG, (B,E) IgM, and (C,F) IgA. Results show the mean+/−SD for n=6 neonatal animals per group and n=9-10 juvenile animals per group. The statistical significance was analyzed with one-way ANOVA followed by Tukey's post test: *P<0.05, **P<0.01, ***P<0.001, ****P<0.0001. Unlabeled comparisons between single- and repeat-dose groups were not significantly different.

FIGS. 9A-9F Quantification of IgG, IgM, and IgA capsid binding antibodies in the BALF of AV.TL65 infected ferrets. (A-F) Quantification of capsid binding antibodies in the BALF of (A-C) neonatal and (D-F) juvenile ferrets for (A,D) IgG, (B,E) IgM, and (C,F) IgA. Results show the mean+/−SD for n=6 neonatal animals per group and n=9-10 juvenile animals per group. The statistical significance was analyzed with one-way ANOVA followed by Tukey's post test: *P<0.05, **P<0.01, ***P<0.001, ****P<0.0001. Unlabeled comparisons between single- and repeat-dose groups were not significantly different.

### DETAILED DESCRIPTION OF EMBODIMENTS OF THE DISCLOSURE

The AV.TL65 capsid protein confers a significant enhancement in apical transduction of airway epithelial cells as compared to other AAV serotypes. As described in Excoffon et al. Proc. Natl. Acad. Sci. USA 106(10):3865-3870, 2009, which is incorporated by reference herein in its entirety, this capsid protein confers at least 10- to 100-fold improvement in expression of the reporter transgene luciferase compared to rAAVs typed with AAV2, AAV5, or AAV9 capsid proteins. The present disclosure is based, at least in part, on the unexpected discovery that transduction and/or expression of transgenes carried by rAAV vectors serotyped with AV.TL65 capsid proteins can be significantly improved to an even greater degree with minimal toxicity by use in combination with one or more augmenters as described herein. For example, as is described in Example 1, combining AV.TL65Luciferase-mCherry with augmenters such as doxorubicin or idarubicin provided non-toxic enhancement of luciferase expression of air-liquid interface (ALI) human bronchial epithelial (HBE) cultures by more than 600-fold compared to AV.TL65Luciferase-mCherry without the augmenter. Thus, the methods described herein allow for high efficiency transduction and expression of transgenes from rAAVs containing AV.TL65 capsid proteins, and find use, for example, in improved methods of treating disorders such as cystic fibrosis. In one aspect, the subject having cystic fibrosis is a human neonate. In one aspect, the subject having cystic fibrosis is a human juvenile. The disclosure also provides pharmaceutical compositions that include (i) an rAAV that includes an AV.TL65 capsid protein and a polynucleotide including a transgene (e.g., CFTRΔR); and (ii) an augmenter of AAV transduction.

### Definitions

The term “AAV” refers to adeno-associated virus, and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA, and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles or, as used herein, “tropism” for different tissue types. As used herein, the term “serotype” refers to an AAV which is identified by and distinguished from other AAVs based on capsid protein reactivity with defined antisera, e.g., AAV1, AAV2, AAV3, AAV4, AAV5, AAV6, AAV7, AAV8, AAV9, and AAVrh10. For example, serotype AAV2 is used to refer to an AAV which contains capsid proteins encoded from the cap gene of AAV2 and a genome containing 5′ and 3′ ITR sequences from the same AAV2 serotype. Pseudotyped AAV as refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art.

The term “about” is used herein to mean a value that is ±10% of the recited value.

As used herein, by “administering” is meant a method of giving a dosage of a composition described herein (e.g., an rAAV, an augmenter, and/or a pharmaceutical composition thereof) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. In some embodiments, a composition described herein is administered in aerosolized particles intratracheally and/or intrabronchially using an atomizer sprayer (e.g., with a MADgic® laryngo-tracheal mucosal atomization device).

The compositions utilized in the methods described herein can also be administered locally or systemically. The method of administration can vary depending on various factors (e.g., the components of the composition being administered and the severity of the condition being treated).

The term “anthracycline” refers to a class of drugs used, e.g., in chemotherapy. Exemplary anthracyclines include doxorubicin, idarubicin, aclarubicin, daunorubicin, epirubicin, valrubicin, and mitoxantrone.

The term “AV.TL65” refers to an evolved chimeric AAV capsid protein that is highly tropic for the human airway. AV.TL65 is described in Excoffon et al. supra, and is also known in the art as AAV2.5T. AV.TL65 is a chimera between AAV2 (a.a. 1-128) and AAV5 (a.a. 129-725) with a substitution based on one point mutation (A581T). The amino acid sequence of the AV.TL65 capsid is shown below:

(SEQ ID NO: 13)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPFGLVEEGAKTAPTGKRIDDHFPKRK
KARTEEDSKPSTSSDAEAGPSGSQQLQIPAQPASSLGADTMSAGGGGPLGD
NNQGADGVGNASGDWHCDSTWMGDRVVTKSTRTWVLPSYNNHQYREIKSGS
VDGSNANAYFGYSTPWGYFDENRFHSHWSPRDWQRLINNYWGFRPRSLRVK
IFNIQVKEVTVQDSTTTIANNLTSTVQVFTDDDYQLPYVVGNGTEGCLPAF
PPQVFTLPQYGYATLNRDNTENPTERSSFFCLEYFPSKMLRTGNNFEFTYN
FEEVPFHSSFAPSQNLFKLANPLVDQYLYRFVSTNNTGGVQFNKNLAGRYA
NTYKNWFPGPMGRTQGWNLGSGVNRASVSAFATTNRMELEGASYQVPPQPN
GMTNNLQGSNTYALENTMIFNSQPANPGTTATYLEGNMLITSESETQPVNR
VAYNVGGQMATNNQSSTTAPTTGTYNLQEIVPGSVWMERDVYLQGPIWAKI
PETGAHFHPSPAMGGFGLKHPPPMMLIKNTPVPGNITSFSDVPVSSFITQY
STGQVTVEMEWELKKENSKRWNPEIQYTNNYNDPQFVDFAPDSTGEYRTTR
IGTRYLTRPL.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

A “gene” refers to a polynucleotide containing at least one open reading frame that is capable of encoding a particular protein after being transcribed and translated.

The term “gene delivery” refers to the introduction of an exogenous polynucleotide into a cell for gene transfer, and may encompass targeting, binding, uptake, transport, localization, replicon integration and expression.

The term “gene transfer” refers to the introduction of an exogenous polynucleotide into a cell which may encompass targeting, binding, uptake, transport, localization and replicon integration, but is distinct from and does not imply subsequent expression of the gene.

The term “gene expression” or “expression” refers to the process of gene transcription, translation, and post-translational modification.

A “helper virus” for AAV refers to a virus that allows AAV (e.g., wild-type AAV) to be replicated and packaged by a mammalian cell. A variety of such helper viruses for AAV are known in the art, including adenoviruses, herpes viruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide).

“Host cells,” “cell lines,” “cell cultures,” “packaging cell line” and other such terms denote eukaryotic cells, e.g., mammalian cells, such as human cells, useful in the present disclosure. These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

“Increased transduction or transduction frequency,” “altered transduction or transduction frequency,” or “enhanced transduction or transduction frequency” refers to an increase in one or more of the activities described above in a treated cell relative to an untreated cell. Agents described herein which increase transduction efficiency may be determined by measuring the effect on one or more transduction activities, which may include measuring the expression of the transgene, measuring the function of the transgene, or determining the number of rAAV vector particles necessary to yield the same transgene effect compared to host cells not treated with the agents. An augmenter described herein may result in an increased transduction or transduction frequency of an rAAV containing an AV.TL65 capsid protein relative to a reference level (e.g., the transduction or transduction frequency of the rAAV in the absence of the augmenter).

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more some. Thus, for example, a 2-fold enrichment is some, 10-fold enrichment is more some, 100-fold enrichment is more some, 1000-fold enrichment is even more some.

As used herein, the term “operable linkage” or “operably linked” refers to a physical or functional juxtaposition of the components so described as to permit them to function in their intended manner. More specifically, for example, two DNA sequences operably linked means that the two DNAs are arranged (cis or trans) in such a relationship that at least one of the DNA sequences is able to exert a physiological effect upon the other sequence. For example, an enhancer and/or a promoter can be operably linked with a transgene (e.g., a therapeutic transgene, such as a CFTRΔR minigene).

“Packaging” as used herein refers to a series of subcellular events that results in the assembly and encapsidation of a viral vector, particularly an AAV vector. Thus, when a suitable vector is introduced into a packaging cell line under appropriate conditions, it can be assembled into a viral particle. Functions associated with packaging of viral vectors, particularly AAV vectors, are described herein and in the art.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof that retains the desired biochemical function of the intact protein. Similarly, references to CFTR, and other such genes for use in gene therapy (typically referred to as “transgenes” to be delivered to a recipient cell), include polynucleotides encoding the intact polypeptide or any fragment or genetically engineered derivative possessing the desired biochemical function.

By “pharmaceutical composition” is meant any composition that contains a therapeutically or biologically active agent (e.g., a polynucleotide comprising a transgene (e.g., a CFTRΔR minigene; see, e.g., Ostedgaard et al. Proc. Natl. Acad. Sci. USA 108(7):2921-6, 2011)), either incorporated into a viral vector (e.g., an rAAV vector) or independent of a viral vector (e.g., incorporated into a liposome, microparticle, or nanoparticle)) that is suitable for administration to a subject. Any of these formulations can be prepared by well-known and accepted methods of art. See, for example, Remington: The Science and Practice of Pharmacy (21st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

The terms “proteasome modulating agent” and “proteasome modulator” refer to an agent or class of agents which alter or enhance rAAV transduction or rAAV transduction frequencies by interacting with, binding to, or altering the function of, and/or trafficking or location of the proteasome. Proteasome modulators may have other cellular functions as described in the art, e.g., such as doxorubicin, a chemotherapy drug. Proteasome modulators of the current disclosure include proteasome inhibitors, e.g., bortezomib, carfilzomib, ixazomib, tripeptidyl aldehydes (Z-LLL or LLnL), agents that inhibit calpains, cathepsins, cysteine proteases, and/or chymotrypsin-like protease activity of proteasomes (see, e.g., Wagner et al., Hum. Gene Ther., 13:1349 (2002); Young et al., J. Virol., 74:3953 (2000); and Seisenberger et al., Science, 294:1029 (2001)).

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

By “recombinant adeno-associated virus (AAV)” or “rAAV vector” is meant a recombinantly-produced AAV or AAV particle that comprises a polynucleotide sequence not of AAV origin (e.g., a polynucleotide comprising a transgene, which may be operably linked to one or more enhancer and/or promoters) to be delivered into a cell, either in vivo, ex vivo, or in vitro. Non-naturally occurring (e.g., chimeric) capsids may be used in the rAAVs described herein, e.g., AV.TL65.

By “reference” is meant any sample, standard, or level that is used for comparison purposes. A “normal reference sample” or a “wild-type reference sample” can be, for example, a sample from a subject not having the disorder (e.g., cystic fibrosis). A “positive reference” sample, standard, or value is a sample, standard, value, or number derived from a subject that is known to have a disorder (e.g., cystic fibrosis), which may be matched to a sample of a subject by at least one of the following criteria: age, weight, disease stage, and overall health.

The terms “subject” and “patient” are used interchangeably herein to refer to any mammal (e.g., a human, a primate, a cat, a dog, a ferret, a cow, a horse, a pig, a goat, a rat, or a mouse). In one embodiment, the subject is a human.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

A “therapeutic gene,” “prophylactic gene,” “target polynucleotide,” “transgene,” “gene of interest” and the like generally refer to a gene or genes to be transferred using a vector. Typically, in the context of the present disclosure, such genes are located within the rAAV vector (which vector is flanked by inverted terminal repeat (ITR) regions and thus can be replicated and encapsidated into rAAV particles). Target polynucleotides can be used in this disclosure to generate rAAV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; (vi) polynucleotides for cancer therapy, such as E1A tumor suppressor genes or p53 tumor suppressor genes for the treatment of various cancers; and (vii) polynucleotides for gene editing (e.g., CRISPR). To effect expression of the transgene in a recipient host cell, it is in one embodiment operably linked to a promoter, either its own or a heterologous promoter. A large number of suitable promoters are known in the art, the choice of which depends on the desired level of expression of the target polynucleotide; whether one desires constitutive expression, inducible expression, cell-specific or tissue-specific expression, etc. The rAAV vector may also contain a selectable marker. Exemplary transgenes include, without limitation, cystic fibrosis transmembrane conductance regulator (CFTR) or derivatives thereof (e.g., a CFTRΔR minigene; see, e.g., Ostedgaard et al. Proc. Natl. Acad. Sci. USA 108(7):2921-6, 2011, which is incorporated by reference herein in its entirety), α-antitrypsin, β-globin, γ-globin, tyrosine hydroxylase, glucocerebrosidase, aryl sulfatase A, factor VIII, dystrophin, erythropoietin, alpha 1-antitrypsin, surfactant protein SP-D, SP-A or SP-C, erythropoietin, or a cytokine, e.g., IFN-alpha, IFNγ, TNF, IL-1, IL-17, or IL-6, or a prophylactic protein that is an antigen such as viral, bacterial, tumor or fungal antigen, or a neutralizing antibody or a fragment thereof that targets an epitope of an antigen such as one from a human respiratory virus, e.g., influenza virus or RSV including but not limited to HBoV protein, influenza virus protein, RSV protein, or SARS protein.

By “therapeutically effective amount” is meant the amount of a composition administered to improve, inhibit, or ameliorate a condition of a subject, or a symptom of a disorder or disease, e.g., cystic fibrosis, in a clinically relevant manner. Any improvement in the subject is considered sufficient to achieve treatment. In one embodiment, an amount sufficient to treat is an amount that reduces, inhibits, or prevents the occurrence or one or more symptoms of cystic fibrosis or is an amount that reduces the severity of, or the length of time during which a subject suffers from, one or more symptoms of cystic fibrosis (e.g., by at least about 10%, about 20%, or about 30%, or by at least about 50%, about 60%, or about 70%, or by at least about 80%, about 90%, about 95%, about 99%, or more, relative to a control subject that is not treated with a composition described herein). An effective amount of the pharmaceutical composition used to practice the methods described herein (e.g., the treatment of cystic fibrosis) varies depending upon the manner of administration and the age, body weight, and general health of the subject being treated. A physician or researcher can decide the appropriate amount and dosage regimen.

“Transduction” or “transducing” as used herein, are terms referring to a process for the introduction of an exogenous polynucleotide, e.g., a transgene in rAAV, into a host cell leading to expression of the polynucleotide, e.g., the transgene in the cell. The process generally includes 1) endocytosis of the AAV after it has bound to a cell surface receptor, 2) escape from endosomes or other intracellular compartments in the cytosol of a cell, 3) trafficking of the viral particle or viral genome to the nucleus, 4) uncoating of the virus particles, and generation of expressible double stranded AAV genome forms, including circular intermediates. The rAAV expressible double stranded form may persist as a nuclear episome or optionally may integrate into the host genome. The alteration of any or a combination of endocytosis of the AAV after it has bound to a cell surface receptor, escape from endosomes or other intracellular compartments to the cytosol of a cell, trafficking of the viral particle or viral genome to the nucleus, or uncoating of the virus particles, and generation of expressive double stranded AAV genome forms, including circular intermediates, may result in altered expression levels or persistence of expression, or altered trafficking to the nucleus, or altered types or relative numbers of host cells or a population of cells expressing the introduced polynucleotide. Altered expression or persistence of a polynucleotide introduced via rAAV can be determined by methods well known to the art including, but not limited to, protein expression, e.g., by ELISA, flow cytometry and Western blot, measurement of DNA and RNA production by hybridization assays, e.g., Northern blots, Southern blots and gel shift mobility assays, or quantitative or non-quantitative reverse transcription, polymerase chain reaction (PCR), or digital droplet PCR assays.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency (e.g., cystic fibrosis), infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent. Treatment may reduce one or more symptoms of a pathological condition. For example, symptoms of cystic fibrosis are known in the art and include, e.g., persistent cough, wheezing, breathlessness, exercise intolerance, repeated lung infections, inflamed nasal passages or stuffy nose, foul-smelling or greasy stools, poor weight gain and growth, intestinal blockage, constipation, elevated salt concentrations in sweat, pancreatitis, and pneumonia. Detecting an improvement in, or the absence of, one or more symptoms of a disorder (e.g., cystic fibrosis), indicates successful treatment.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a transgene, may comprise a coding sequence of interest in gene therapy (such as a gene encoding a protein of therapeutic or interest), a coding sequence of interest in vaccine development (such as a polynucleotide expressing a protein, polypeptide or peptide suitable for eliciting an immune response in a mammal), and/or a selectable or detectable marker.

### Recombinant AAV Vectors and Polynucleotides

Recombinant AAV vectors are potentially powerful tools for human gene therapy, particularly for diseases such as cystic fibrosis and sickle cell anemia. A major advantage of rAAV vectors over other approaches to gene therapy is that they generally do not require ongoing replication of the target cell in order to exist episomally or become stably integrated into the host cell. Provided herein are rAAVs that include an AV.TL65 capsid protein and a polynucleotide comprising a transgene, which may be combined with augmenters of AAV transduction, as described herein.

rAAV vectors and/or viruses are also potentially powerful for the development of therapeutic or prophylactic vaccines to prevent infection, progression, and/or severity of disease. A major advantage of rAAV vectors for vaccine development is that they are capable of persisting for essentially the lifetime of the cell as a nuclear episome and therefore provide long term expression of the peptide, polypeptide, or protein of immunologic interest. Transgenes of interest include viral gene e.g. the envelope (env) or gag genes of HIV; bacterial genes e.g., streptococcal cell wall proteins; fungi, e.g., cocidomycosis; parasites, e.g., Leischmaniosis, or cancer genes, e.g. p53.

rAAV vectors and/or viruses may also contain one or more detectable markers. A variety of such markers are known, including, by way of illustration, the bacterial beta-galactosidase (lacZ) gene; the human placental alkaline phosphatase (AP) gene and genes encoding various cellular surface markers which have been used as reporter molecules both in vitro and in vivo. The rAAV vectors and/or viruses may also contain one or more selectable markers.

Recombinant AAV vectors and/or viruses can also comprise polynucleotides that do not encode proteins, including, e.g., polynucleotides encoding for antisense mRNA (the complement of mRNA) which can be used to block the translation of normal mRNA by forming a duplex with it, and polynucleotides that encode ribozymes (RNA catalysts).

An AAV vector typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and are some when it is desired that the therapeutic or prophylactic polynucleotide be expressed on an ongoing basis. Inducible promoters generally exhibit low activity in the absence of the inducer, and are up-regulated in the presence of the inducer. They may be some when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (MMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will likely also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present disclosure.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters are some because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1 CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), optionally linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Given the relative encapsidation size limits of various AAV genomes, insertion of a large heterologous polynucleotide into the genome necessitates removal of a portion of the AAV sequence. Removal of one or more AAV genes is in any case desirable, to reduce the likelihood of generating replication-competent AAV (“RCA”). Accordingly, encoding or promoter sequences for rep, cap, or both, are in one embodiment removed, since the functions provided by these genes can be provided in trans.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this disclosure in a number of different forms. Viral particles, plasmids, and stably transformed host cells can all be used to introduce such constructs into the packaging cell, either transiently or stably.

In certain embodiments of this disclosure, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (e.g., inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this disclosure. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this disclosure.

Approaches for producing rAAVs that contain AV.TL65 capsid proteins are known in the art. See, e.g., Excoffon et al. Proc. Natl. Acad. Sci. USA 106(10):3865-3870, 2009 and U.S. Pat. No. 10,046,016, each of which is incorporated herein by reference in its entirety. In some embodiments, the polynucleotide may contain any of the enhancers or promoters described in U.S. patent application Ser. No. 16/082,767, which is incorporated by reference herein in its entirety.

The rAAV may include a polynucleotide containing any of the enhancers and/or promoters described herein or known in the art. For example, the rAAV may include a polynucleotide including an F5 enhancer and/or a tg83 promoter. In some embodiments, the F5 enhancer includes the polynucleotide sequence of SEQ ID NO:1 or SEQ ID NO:14, or a variant thereof with at least 80% nucleic acid sequence identity to SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14. In some embodiments, the tg83 promoter includes the polynucleotide sequence of SEQ ID NO:2 or a variant thereof with at least 80% nucleic acid sequence identity to SEQ ID NO:2.

The rAAV may include any suitable transgene. In some embodiments, the transgene is CFTR or a derivative thereof. In some embodiments, the derivative of CFTR is a CFTRΔR transgene (e.g., a human CFTRΔR transgene). In some embodiments, the human CFTRΔR transgene is encoded by a polynucleotide including the sequence of SEQ ID NO:4, or a variant thereof with at least 80% nucleic acid sequence identity to SEQ ID NO:4.

In some embodiments, the polynucleotide includes, in a 5′-to-3′ direction, the F5 enhancer, the tg83 promoter, and the CFTRΔR transgene. For example, in some embodiments, the polynucleotide includes the sequence of SEQ ID NO:7, or a variant thereof with at least 80% nucleic acid sequence identity to SEQ ID NO:7.

The polynucleotide may further include, in the 3′ direction, a 3′ untranslated region (3′-UTR) including the sequence of SEQ ID NO:5, or a variant thereof with at least 80% nucleic acid sequence identity to SEQ ID NO:5.

The polynucleotide may further include, in the 3′ direction, a synthetic polyadenylation site including the sequence of SEQ ID NO:6, or a variant thereof with at least 80% nucleic acid sequence identity to SEQ ID NO:6.

The polynucleotide may further include one or more ITRs, e.g., a 5′ adeno-associated virus (AAV) inverted terminal repeat (ITR) at the 5′ terminus of the polynucleotide and a 3′ AAV ITR at the 3′ terminus of the polynucleotide. Any suitable 5′ ITR and/or 3′ ITR may be used. In some embodiments, the 5′ AAV ITR includes the sequence of SEQ ID NO:15, or a variant thereof with at least 80% nucleic acid sequence identity to SEQ ID NO:15. In some embodiments, the 3′ AAV ITR includes the sequence of SEQ ID NO:16, or a variant thereof with at least 80% nucleic acid sequence identity to SEQ ID NO:16. The ITR sequences may be palindromic, e.g., as in SEQ ID NO:15 and SEQ ID NO:16, where the ITR sequence on the 5′ end is located on the reverse strand, and the ITR sequence on the 3′ end is located on the forward strand.

In some examples, the polynucleotide comprises: a 5′ AAV ITR including the sequence of SEQ ID NO:15, an F5 enhancer including the sequence of SEQ ID NO:14 (which may include a 5′ EcoRI site and a 3′ XhoI site, as in SEQ ID NO:1), a tg83 promoter including the sequence of SEQ ID NO:2, a 5′ UTR comprising the sequence of SEQ ID NO:3, a hCFTRΔR transgene including the sequence of SEQ ID NO:4, a 3′ UTR comprising the sequence of SEQ ID NO:5, a s-pA including the sequence of SEQ ID NO:6, and a 3′ AAV ITR comprising the sequence of SEQ ID NO:16.

In particular examples, the polynucleotide includes the sequence of SEQ ID NO:17, or a variant thereof with at least 80% nucleic acid sequence identity to SEQ ID NO:17.

Uses of rAAV and Pharmaceutical Compositions Thereof for Gene Therapy

AAV vectors can be used for administration to an individual for purposes of gene therapy or vaccination. Suitable diseases for rAAV therapy include but are not limited to those induced by viral, bacterial, or parasitic infections, various malignancies and hyperproliferative conditions, autoimmune conditions, and congenital deficiencies (e.g., cystic fibrosis).

Gene therapy can be conducted to enhance the level of expression of a particular protein either within or secreted by the cell. Vectors described herein may be used to genetically alter cells either for gene marking, replacement of a missing or defective gene, or insertion of a therapeutic gene. Alternatively, a polynucleotide may be provided to the cell that decreases the level of expression. This may be used for the suppression of an undesirable phenotype, such as the product of a gene amplified or overexpressed during the course of a malignancy, or a gene introduced or overexpressed during the course of a microbial infection. Expression levels may be decreased by supplying a therapeutic or prophylactic polynucleotide comprising a sequence capable, for example, of forming a stable hybrid with either the target gene or RNA transcript (antisense therapy), capable of acting as a ribozyme to cleave the relevant mRNA or capable of acting as a decoy for a product of the target gene.

Of particular interest is the correction of the genetic defect of cystic fibrosis, by supplying a properly functioning cystic fibrosis transmembrane conductance regulator (CFTR) to the airway epithelium. Thus, rAAV vectors encoding native CFTR protein, and mutants and fragments thereof, e.g., CFTRΔR, and pharmaceutical compositions thereof, are all some embodiments of this disclosure.

The disclosure provides a pharmaceutical composition that includes (i) an rAAV that includes an AV.TL65 capsid protein and a polynucleotide comprising a transgene (e.g., CFTRΔR); and (ii) an augmenter of AAV transduction. In some embodiments, the augmenter is a proteasome modulating agent. In some embodiments, the proteasome modulating agent is an anthracycline, a proteasome inhibitor, a tripeptidyl aldehyde, or a combination thereof. In some embodiments, the anthracycline is doxorubicin, idarubicin, aclarubicin, daunorubicin, epirubicin, valrubicin, mitoxantrone, or a combination thereof. In some embodiments, the anthracycline is doxorubicin, idarubicin, or a combination thereof. In some embodiments, the proteasome inhibitor is bortezomib, carfilzomib, and ixazomib. In some embodiments, the tripeptidyl aldehyde is N-acetyl-1-leucyl-1-leucyl-1-norleucine (LLnL).

The rAAV of the pharmaceutical composition may include a polynucleotide containing any of the enhancers and/or promoters described herein or known in the art. For example, the rAAV may include a polynucleotide including an F5 enhancer and/or a tg83 promoter. In some embodiments, the F5 enhancer includes the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14. In some embodiments, the tg83 promoter includes the polynucleotide sequence of SEQ ID NO:2, or a variant thereof with at least 80% nucleic acid sequence identity to SEQ ID NO:2.

The rAAV may include any suitable transgene. In some embodiments, the transgene is CFTR or a derivative thereof. In some embodiments, the derivative of CFTR is a CFTRΔR transgene (e.g., a human CFTRΔR transgene). In some embodiments, the human CFTRΔR transgene is encoded by a polynucleotide including the sequence of SEQ ID NO:4, or a variant thereof with at least 80% nucleic acid sequence identity to SEQ ID NO:4.

In some embodiments, the polynucleotide includes, in a 5′-to-3′ direction, the F5 enhancer, the tg83 promoter, and the CFTRΔR transgene. For example, in some embodiments, the polynucleotide includes the sequence of SEQ ID NO:7, or a variant thereof with at least 80% nucleic acid sequence identity to SEQ ID NO:7.

The polynucleotide may further include, in the 3′ direction, a 3′-UTR including the sequence of SEQ ID NO:5, or a variant thereof with at least 80% nucleic acid sequence identity to SEQ ID NO:5.

The polynucleotide may further include, in the 3′ direction, a synthetic polyadenylation site including the sequence of SEQ ID NO:6, or a variant thereof with at least 80% nucleic acid sequence identity to SEQ ID NO:6.

The polynucleotide may further include one or more ITRs, e.g., a 5′ adeno-associated virus (AAV) inverted terminal repeat (ITR) at the 5′ terminus of the polynucleotide and a 3′ AAV ITR at the 3′ terminus of the polynucleotide. Any suitable 5′ ITR and/or 3′ ITR may be used. In some embodiments, the 5′ AAV ITR includes the sequence of SEQ ID NO:15. In some embodiments, the 3′ AAV ITR includes the sequence of SEQ ID NO:16, or a variant thereof with at least 80% nucleic acid sequence identity to SEQ ID NO:16.

In some examples, the polynucleotide includes: a 5′ AAV ITR including the sequence of SEQ ID NO:15, an F5 enhancer including the sequence of SEQ ID NO:14 (which may include a 5′ EcoRI site and a 3′ XhoI site, as in SEQ ID NO:1), a tg83 promoter including the sequence of SEQ ID NO:2, a 5′ UTR including the sequence of SEQ ID NO:3, a hCFTRΔR transgene including the sequence of SEQ ID NO:4, a 3′ UTR comprising the sequence of SEQ ID NO:5, a s-pA including the sequence of SEQ ID NO:6, and a 3′ AAV ITR including the sequence of SEQ ID NO:16.

In particular examples, the polynucleotide includes the sequence of SEQ ID NO:17, or a variant thereof with at least 80% nucleic acid sequence identity to SEQ ID NO:17.

Compositions described herein (e.g., rAAVs, pharmaceutical compositions, and/or augmenters) may be used in vivo as well as ex vivo. In vivo gene therapy comprises administering the vectors of this disclosure directly to a subject. Pharmaceutical compositions can be supplied as liquid solutions or suspensions, as emulsions, or as solid forms suitable for dissolution or suspension in liquid prior to use. For administration into the respiratory tract, one exemplary mode of administration is by aerosol, using a composition that provides either a solid or liquid aerosol when used with an appropriate aerosolubilizer device. Another some mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

A composition described herein (e.g., rAAVs, pharmaceutical compositions, and/or augmenters) can be administered by any suitable route, e.g., by inhalation, nebulization, aerosolization, intranasally, intratracheally, intrabronchially, orally, parenterally (e.g., intravenously, subcutaneously, or intramuscularly), orally, nasally, rectally, topically, or buccally. They can also be administered locally or systemically. In some embodiments, a composition described herein is administered in aerosolized particles intratracheally and/or intrabronchially using an atomizer sprayer (e.g., with a MADgic® laryngo-tracheal mucosal atomization device). In some embodiments, the pharmaceutical composition is administered parentally. In other some embodiments, the pharmaceutical composition is administered systemically. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

For administration to the upper (nasal) or lower respiratory tract by inhalation, the compositions described herein (e.g., rAAVs, pharmaceutical compositions, and/or augmenters) are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

Administration of the compositions described herein (e.g., rAAVs, pharmaceutical compositions, and/or augmenters) may be continuous or intermittent, depending, for example, upon the recipient's physiological condition, whether the purpose of the administration is therapeutic or prophylactic, and other factors known to skilled practitioners. The rAAVs or pharmaceutical compositions described herein can be administered once, or multiple times, at the same or at different sites. The administration of the agents of the disclosure may be essentially continuous over a preselected period of time or may be in a series of spaced doses.

The compositions described herein (e.g., rAAVs, pharmaceutical compositions, and/or augmenters) can be administered in combination with one or more additional therapeutic agent. Any suitable additional therapeutic agent(s) may be used, including standard of care therapies for CF. In some embodiments, the one or more additional therapeutic agents includes an antibiotic (e.g., azithromycin (ZITHROMAX®), amoxicillin and clavulanic acid (AUGMENTIN®), cloxacillin and diclocacillin, ticarcillin and clavulanic acid (TIMENTIN®), cephalexin, cefdinir, cefprozil, cefaclor; sulfamethoxazole and trimethoprim (BACTRIM®), erythromycin/sulfisoxazole, erythromycin, clarithromycin, tetracycline, doxycycline, minocycline, tigecycline, vancomycin, imipenem, meripenem, Colistimethate/COLISTIN®, linezolid, ciprofloxacin, levofloxacin, or a combination thereof), a mucus thinner (e.g., hypertonic saline or dornase alfa (PULMOZYME®)), a CFTR modulator (e.g., ivacaftor (KALYDECO®), lumacaftor, lumacaftor/ivacaftor (ORKAMBI®), tezacaftor/ivacaftor (SYMDEKO®), or TRIKAFTA® (elexacaftor/ivacaftor/tezacaftor)), a mucolytic (e.g., acetylcysteine, ambroxol, bromhexine, carbocisteine, erdosteine, mecysteine, and dornase alfa), an immunosuppressive agent, normal saline, hypertonic saline, or a combination thereof.

For example, any one the compositions described herein (e.g., rAAVs, pharmaceutical compositions, and/or augmenters) may be administered in combination with one or more immunosuppressive agents. Any suitable immunosuppressive agent may be used. For example, non-limiting examples of immunosuppressive agents include corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone), polyclonal anti-lymphocyte antibodies (e.g., anti-lymphocyte globulin (ALG) and anti-thymocyte globulin (ATG) antibodies, which may be, for example, horse- or rabbit-derived), monoclonal anti-lymphocyte antibodies (e.g., anti-CD3 antibodies (e.g., murmomab and alemtuzumab) or anti-CD20 antibodies (e.g., rituximab)), interleukin-2 (IL-2) receptor antagonists (e.g., daclizumab and basiliximab), calcineurin inhibitors (e.g., cyclosporin A and tacrolimus), cell cycle inhibitors (e.g., azathioprine, mycophenolate mofetil (MMF), and mycophenolic acid (MPA)), mammalian target of rapamycin (mTOR) inhibitors (e.g., sirolimus (rapamycin) and everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, and a corticosteroid).

In particular embodiments, any one the compositions described herein (e.g., rAAVs, pharmaceutical compositions, and/or augmenters) may be administered in combination with one or more corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone).

An immunosuppressive agent (e.g., any immunosuppressive agent described herein) may be administered by inhalation or administered systemically (e.g., intravenously or subcutaneously).

The compositions described herein (e.g., rAAVs, pharmaceutical compositions, and/or augmenters) may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present compositions will vary with the form of administration, the particular compound chosen and the physiological characteristics of the particular patient under treatment. It is desirable that the lowest effective concentration of virus be utilized in order to reduce the risk of undesirable effects, such as toxicity.

### Augmenters

As described herein, rAAVs containing AV.TL65 capsid proteins can be used in combination with augmenters of AAV transduction to achieve significant increases in transduction and/or expression of transgenes. Any suitable augmenter can be used. For example, U.S. Pat. No. 7,749,491, which is incorporated by reference herein in its entirety, describes suitable augmenters. The augmenter may be a proteasome modulating agent. The proteasome modulating agent may be an anthracycline (e.g., doxorubicin, idarubicin, aclarubicin, daunorubicin, epirubicin, valrubicin, or mitoxantrone), a proteasome inhibitor (e.g., bortezomib, carfilzomib, and ixazomib), a tripeptidyl aldehyde (e.g., N-acetyl-1-leucyl-1-leucyl-1-norleucine (LLnL)), or a combination thereof. In some embodiments, the augmenter is doxorubicin. In other embodiments, the augmenter is idarubicin.

The rAAV and the augmenter(s) may be contacted with a cell, or administered to a subject, in the same composition or in different compositions (e.g., pharmaceutical compositions). The contacting or the administration of the rAAV and the augmenter(s) may be sequential (e.g., rAAV followed by the augmenter(s), or vice versa) or simultaneous.

### EXAMPLES

The disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Delivery of AAV-CFTR to Bronchial Epithelial Cells from Cystic Fibrosis Patients Augments Functional Recovery of Chloride Conductance

Cystic fibrosis (CF) is a life-threatening, autosomal recessive disease caused by mutations in the gene encoding the cystic fibrosis transmembrane conductance regulator (CFTR), a channel that conducts chloride and bicarbonate ions across epithelial cell membranes. Impaired CFTR function leads to inflammation of the airways and progressive bronchiectasis. Because of the single-gene etiology of CF and the various CFTR mutations in the patient population, gene therapy potentially provides a universal cure for CF. The standard of care for CF currently attempts to modulate the activity of defective CFTR using modulators, for example, lumacaftor/VX-809 (a channel corrector), ivacaftor/VX-770 (a channel potentiator) ORKAMBI® (a combination of the drugs), or TRIKAFTA® (elexacaftor/ivacaftor/tezacaftor). While these approaches are promising, they are limited by their specificity for only subsets of the known CFTR mutations.

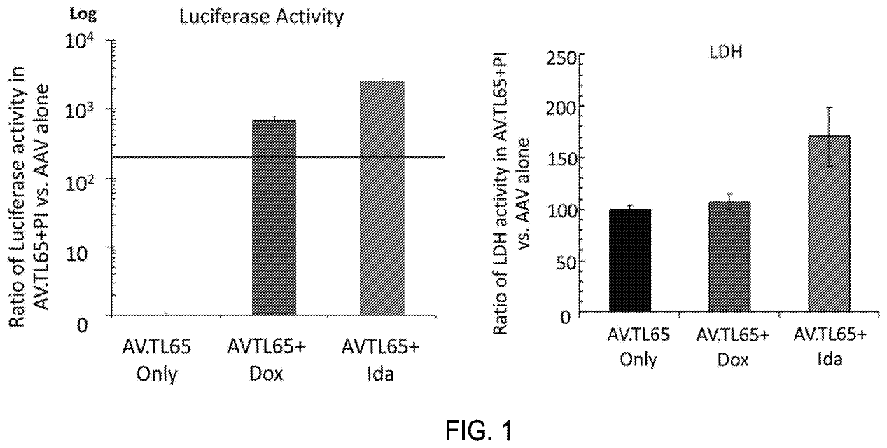

We have generated a novel AAV vector featuring a capsid that is highly efficient at transducing human airway epithelium in the apical membrane. Specifically, we have used AV.TL65-SP183-CFTRΔR to deliver an R-domain-partially-deleted CFTR minigene and AV.TL65Luciferase-mCherry, a dual reporter vector, to express luciferase and fluorescent mCherry protein. The AV.TL65-SP183-CFTRΔR rAAV vector included a polynucleotide comprising: a 5′ AAV ITR comprising the sequence of SEQ ID NO:15, an F5 enhancer comprising the sequence of SEQ ID NO:14 (which may include a 5′ EcoRI site and a 3′ XhoI site, as in SEQ ID NO:1), a tg83 promoter comprising the sequence of SEQ ID NO:2, a 5′ UTR comprising the sequence of SEQ ID NO:3, a hCFTRΔR minigene comprising the sequence of SEQ ID NO:4, a 3′ UTR comprising the sequence of SEQ ID NO:5, a s-pA comprising the sequence of SEQ ID NO:6, and a 3′ AAV ITR comprising the sequence of SEQ ID NO:16. For example, the packaged polynucleotide may include the sequence of SEQ ID NO:17. We have also made use of small molecule augmenters (proteasome inhibitors) to significantly enhance recombinant AAV transduction by stimulating endosomal processing and nuclear trafficking of the viral transgene. We have shown that combining AV.TL65Luciferase-mCherry with doxorubicin or idarubicin provides non-toxic enhancement of luciferase expression by more than 600-fold of air-liquid interface (ALI) human bronchial epithelial (HBE) cultures from 5 separate CF (homozygous dF508/dF508 CFTR) and non-CF donors compared to AV.TL65Luciferase-mCherry without proteasome inhibitor. In another experiment, doxorubicin and idarubicin+AV.TL65-gLuc-mCherry improved transduction over AAV without a proteasome inhibitor by over 200-fold (FIG. 1A, dashed line). Doxorubicin added to AV.TL65-gLuc-mCherry but not idarubicin had less than 150% LDH (toxicity) activity compared to AAV without a proteasome inhibitor (FIG. 1B; dashed line). Transduction efficiency gains in passage 0 (PO) cells were well above 200-fold better with doxorubicin and idarubicin with LDH <1.5× baseline for doxorubicin and >1.5× baseline for idarubicin.

We have also shown that AV.TL65-SP183-CFTROR, when paired with doxorubicin or idarubicin, yields a mean correction of forskolin-stimulated, CFTR-mediated chloride transport in ALI HBE cultures from 6 separate CF donors that is a least 104% that of 6 separate non-CF donors. Furthermore, we have shown this complementation of forskolin-stimulated current is up to four times greater than the standard of care treatment drugs, lumacaftor and ivacaftor, in ALI HBE cultures from two separate HBE CF cell donor lines. In summary, we have developed a method to augment CFTR expression using an AAV viral vector to correct chloride channel defects in HBE cells from CF patients.

SEQUENCE LISTING

| SEQ | | |
|---|---|---|
| ID | | |
| NO | Name | Sequence |
| 1 | F5 | GAATTCGTGGTGAGCGTCTGGGCATGTCTGGGCATGTCTGG |
| | Enhancer | GCATGTCTGGGCATGTCGGGCATTCTGGGCGTCTGGGCATG |
| | with 5′ | TCTGGGCATGTCTGGGCATCTCGAG |
| | EcoRI | |
| | and 3′ | |
| | Xhol | |
| | sites | |
| 2 | tg83 | AACGGTGACGTGCACGCGTGGGCGGAGCCATCACGCAGGT |
| | Promoter | TGCTATATAAGCAGAGCTCGTTTAGTGAACCGTCAGA |
| 3 | 5′-UTR | GTCGAGCCCGAGAGACC |
| 4 | hCFTRΔR | ATGCAGAGGTCGCCTCTGGAAAAGGCCAGCGTTGTCTCCAA |
| | | ACTTTTTTTCAGCTGGACCAGACCAATTTTGAGGAAAGGATA |
| | | CAGACAGCGCCTGGAATTGTCAGACATATACCAAATCCCTTC |
| | | TGTTGATTCTGCTGACAATCTATCTGAAAAATTGGAAAGAGAA |
| | | TGGGATAGAGAGCTGGCTTCAAAGAAAAATCCTAAACTCATT |
| | | AATGCCCTTCGGCGATGTTTTTTCTGGAGATTTATGTTCTATG |
| | | GAATCTTTTTATATTTAGGGGAAGTCACCAAAGCAGTACAGC |
| | | CTCTCTTACTGGGAAGAATCATAGCTTCCTATGACCCGGATA |
| | | ACAAGGAGGAACGCTCTATCGCGATTTATCTAGGCATAGGCT |
| | | TATGCCTTCTCTTTATTGTGAGGACACTGCTCCTACACCCAG |
| | | CCATTTTTGGCCTTCATCACATTGGAATGCAGATGAGAATAG |
| | | CTATGTTTAGTTTGATTTATAAGAAGACTTTAAAGCTGTCAAG |
| | | CCGTGTTCTAGATAAAATAAGTATTGGACAACTTGTTAGTCTC |
| | | CTTTCCAACAACCTGAACAAATTTGATGAAGGACTTGCATTG |
| | | GCACATTTCGTGTGGATCGCTCCTTTGCAAGTGGCACTCCTC |
| | | ATGGGGCTAATCTGGGAGTTGTTACAGGCGTCTGCCTTCTGT |
| | | GGACTTGGTTTCCTGATAGTCCTTGCCCTTTTTCAGGCTGGG |
| | | CTAGGGAGAATGATGATGAAGTACAGAGATCAGAGAGCTGG |
| | | GAAGATCAGTGAAAGACTTGTGATTACCTCAGAAATGATCGA |
| | | GAACATCCAATCTGTTAAGGCATACTGCTGGGAAGAAGCAAT |
| | | GGAAAAAATGATTGAAAACTTAAGACAAACAGAACTGAAACT |
| | | GACTCGGAAGGCAGCCTATGTGAGATACTTCAATAGCTCAGC |
| | | CTTCTTCTTCTCAGGGTTCTTTGTGGTGTTTTTATCTGTGCTT |
| | | CCCTATGCACTAATCAAAGGAATCATCCTCCGGAAAATATTC |
| | | ACCACCATCTCATTCTGCATTGTTCTGCGCATGGCGGTCACT |
| | | CGGCAATTTCCCTGGGCTGTACAAACATGGTATGACTCTCTT |
| | | GGAGCAATAAACAAAATACAGGATTTCTTACAAAAGCAAGAA |
| | | TATAAGACATTGGAATATAACTTAACGACTACAGAAGTAGTGA |
| | | TGGAGAATGTAACAGCCTTCTGGGAGGAGGGATTTGGGGAA |
| | | TTATTTGAGAAAGCAAAACAAAACAATAACAATAGAAAAACTT |
| | | CTAATGGTGATGACAGCCTCTTCTTCAGTAATTTCTCACTTCT |
| | | TGGTACTCCTGTCCTGAAAGATATTAATTTCAAGATAGAAAGA |
| | | GGACAGTTGTTGGCGGTTGCTGGATCCACTGGAGCAGGCAA |
| | | GACTTCACTTCTAATGATGATTATGGGAGAACTGGAGCCTTC |
| | | AGAGGGTAAAATTAAGCACAGTGGAAGAATTTCATTCTGTTC |
| | | TCAGTTTTCCTGGATTATGCCTGGCACCATTAAAGAAAATATC |
| | | ATCTTTGGTGTTTCCTATGATGAATATAGATACAGAAGCGTCA |
| | | TCAAAGCATGCCAACTAGAAGAGGACATCTCCAAGTTTGCAG |
| | | AGAAAGACAATATAGTTCTTGGAGAAGGTGGAATCACACTGA |
| | | GTGGAGGTCAACGAGCAAGAATTTCTTTAGCAAGAGCAGTAT |
| | | ACAAAGATGCTGATTTGTATTTATTAGACTCTCCTTTTGGATA |
| | | CCTAGATGTTTTAACAGAAAAAGAAATATTTGAAAGCTGTGTC |
| | | TGTAAACTGATGGCTAACAAAACTAGGATTTTGGTCACTTCTA |
| | | AAATGGAACATTTAAAGAAAGCTGACAAAATATTAATTTTGCA |
| | | TGAAGGTAGCAGCTATTTTTATGGGACATTTTCAGAACTCCAA |
| | | AATCTACAGCCAGACTTTAGCTCAAAACTCATGGGATGTGAT |
| | | TCTTTCGACCAATTTAGTGCAGAAAGAAGAAATTCAATCCTAA |
| | | CTGAGACCTTACACCGTTTCTCATTAGAAGGAGATGCTCCTG |
| | | TCTCCTGGACAGAAACAAAAAAACAATCTTTTAAACAGACTG |
| | | GAGAGTTTGGGGAAAAAAGGAAGAATTCTATTCTCAATCCAA |
| | | TCAACTCTACGCTTCAGGCACGAAGGAGGCAGTCTGTCCTG |
| | | AACCTGATGACACACTCAGTTAACCAAGGTCAGAACATTCAC |
| | | CGAAAGACAACAGCATCCACACGAAAAGTGTCACTGGCCCC |
| | | TCAGGCAAACTTGACTGAACTGGATATATATTCAAGAAGGTT |
| | | ATCTCAAGAAACTGGCTTGGAAATAAGTGAAGAAATTAACGA |
| | | AGAAGACTTAAAGGAGTGCCTTTTTGATGATATGGAGAGCAT |
| | | ACCAGCAGTGACTACATGGAACACATACCTTCGATATATTAC |
| | | TGTCCACAAGAGCTTAATTTTTGTGCTAATTTGGTGCTTAGTA |
| | | ATTTTTCTGGCAGAGGTGGCTGCTTCTTTGGTTGTGCTGTGG |
| | | CTCCTTGGAAACACTCCTCTTCAAGACAAAGGGAATAGTACT |
| | | CATAGTAGAAATAACAGCTATGCAGTGATTATCACCAGCACC |
| | | AGTTCGTATTATGTGTTTTACATTTACGTGGGAGTAGCCGAC |
| | | ACTTTGCTTGCTATGGGATTCTTCAGAGGTCTACCACTGGTG |
| | | CATACTCTAATCACAGTGTCGAAAATTTTACACCACAAAATGT |
| | | TACATTCTGTTCTTCAAGCACCTATGTCAACCCTCAACACGTT |
| | | GAAAGCAGGTGGGATTCTTAATAGATTCTCCAAAGATATAGC |
| | | AATTTTGGATGACCTTCTGCCTCTTACCATATTTGACTTCATC |
| | | CAGTTGTTATTAATTGTGATTGGAGCTATAGCAGTTGTCGCA |
| | | GTTTTACAACCCTACATCTTTGTTGCAACAGTGCCAGTGATA |
| | | GTGGCTTTTATTATGTTGAGAGCATATTTCCTCCAAACCTCAC |
| | | AGCAACTCAAACAACTGGAATCTGAAGGCAGGAGTCCAATTT |
| | | TCACTCATCTTGTTACAAGCTTAAAAGGACTATGGACACTTCG |
| | | TGCCTTCGGACGGCAGCCTTACTTTGAAACTCTGTTCCACAA |
| | | AGCTCTGAATTTACATACTGCCAACTGGTTCTTGTACCTGTCA |
| | | ACACTGCGCTGGTTCCAAATGAGAATAGAAATGATTTTTGTC |
| | | ATCTTCTTCATTGCTGTTACCTTCATTTCCATTTTAACAACAG |
| | | GAGAAGGAGAAGGAAGAGTTGGTATTATCCTGACTTTAGCCA |
| | | TGAATATCATGAGTACATTGCAGTGGGCTGTAAACTCCAGCA |
| | | TAGATGTGGATAGCTTGATGCGATCTGTGAGCCGAGTCTTTA |
| | | AGTTCATTGACATGCCAACAGAAGGTAAACCTACCAAGTCAA |
| | | CCAAACCATACAAGAATGGCCAACTCTCGAAAGTTATGATTA |
| | | TTGAGAATTCACACGTGAAGAAAGATGACATCTGGCCCTCAG |
| | | GGGGCCAAATGACTGTCAAAGATCTCACAGCAAAATACACAG |
| | | AAGGTGGAAATGCCATATTAGAGAACATTTCCTTCTCAATAAG |
| | | TCCTGGCCAGAGGGTGGGCCTCTTGGGAAGAACTGGATCAG |
| | | GGAAGAGTACTTTGTTATCAGCTTTTTTGAGACTACTGAACAC |
| | | TGAAGGAGAAATCCAGATCGATGGTGTGTCTTGGGATTCAAT |
| | | AACTTTGCAACAGTGGAGGAAAGCCTTTGGAGTGATACCACA |
| | | GAAAGTATTTATTTTTTCTGGAACATTTAGAAAAAACTTGGAT |
| | | CCCTATGAACAGTGGAGTGATCAAGAAATATGGAAAGTTGCA |
| | | GATGAGGTTGGGCTCAGATCTGTGATAGAACAGTTTCCTGG |
| | | GAAGCTTGACTTTGTCCTTGTGGATGGGGGCTGTGTCCTAAG |
| | | CCATGGCCACAAGCAGTTGATGTGCTTGGCTAGATCTGTTCT |
| | | CAGTAAGGCGAAGATCTTGCTGCTTGATGAACCCAGTGCTCA |
| | | TTTGGATCCAGTAACATACCAAATAATTAGAAGAACTCTAAAA |
| | | CAAGCATTTGCTGATTGCACAGTAATTCTCTGTGAACACAGG |
| | | ATAGAAGCAATGCTGGAATGCCAACAATTTTTGGTCATAGAA |
| | | GAGAACAAAGTGCGGCAGTACGATTCCATCCAGAAACTGCT |
| | | GAACGAGAGGAGCCTCTTCCGGCAAGCCATCAGCCCCTCCG |
| | | ACAGGGTGAAGCTCTTTCCCCACCGGAACTCAAGCAAGTGC |
| | | AAGTCTAAGCCCCAGATTGCTGCTCTGAAAGAGGAGACAGA |
| | | AGAAGAGGTGCAAGATACAAGGCTTTAG |
| 5 | 3′-UTR | AGAGCAGCATAAATGTTGACATGGGACATTTGCTCATGGAAT |
| | | TGG |
| 6 | s-pA | AATAAAGAGCTCAGATGCATCGATCAGAGTGTGTTGGTTTTT |
| | | TGTGTGTA |
| 7 | F5 | GAATTCGTGGTGAGCGTCTGGGCATGTCTGGGCATGTCTGG |
| | Enhancer, | GCATGTCTGGGCATGTCGGGCATTCTGGGCGTCTGGGCATG |
| | Tg83 | TCTGGGCATGTCTGGGCATCTCGAGAACGGTGACGTGCACG |
| | Promoter, | CGTGGGCGGAGCCATCACGCAGGTTGCTATATAAGCAGAGC |
| | 5′-UTR, | TCGTTTAGTGAACCGTCAGAGTCGAGCCCGAGAGACCATGC |
| | hCFTRΔR | AGAGGTCGCCTCTGGAAAAGGCCAGCGTTGTCTCCAAACTTT |
| | | TTTTCAGCTGGACCAGACCAATTTTGAGGAAAGGATACAGAC |
| | | AGCGCCTGGAATTGTCAGACATATACCAAATCCCTTCTGTTG |
| | | ATTCTGCTGACAATCTATCTGAAAAATTGGAAAGAGAATGGG |
| | | ATAGAGAGCTGGCTTCAAAGAAAAATCCTAAACTCATTAATG |
| | | CCCTTCGGCGATGTTTTTTCTGGAGATTTATGTTCTATGGAAT |
| | | CTTTTTATATTTAGGGGAAGTCACCAAAGCAGTACAGCCTCT |
| | | CTTACTGGGAAGAATCATAGCTTCCTATGACCCGGATAACAA |
| | | GGAGGAACGCTCTATCGCGATTTATCTAGGCATAGGCTTATG |
| | | CCTTCTCTTTATTGTGAGGACACTGCTCCTACACCCAGCCAT |
| | | TTTTGGCCTTCATCACATTGGAATGCAGATGAGAATAGCTAT |
| | | GTTTAGTTTGATTTATAAGAAGACTTTAAAGCTGTCAAGCCGT |
| | | GTTCTAGATAAAATAAGTATTGGACAACTTGTTAGTCTCCTTT |
| | | CCAACAACCTGAACAAATTTGATGAAGGACTTGCATTGGCAC |
| | | ATTTCGTGTGGATCGCTCCTTTGCAAGTGGCACTCCTCATGG |
| | | GGCTAATCTGGGAGTTGTTACAGGCGTCTGCCTTCTGTGGA |
| | | CTTGGTTTCCTGATAGTCCTTGCCCTTTTTCAGGCTGGGCTA |
| | | GGGAGAATGATGATGAAGTACAGAGATCAGAGAGCTGGGAA |
| | | GATCAGTGAAAGACTTGTGATTACCTCAGAAATGATCGAGAA |
| | | CATCCAATCTGTTAAGGCATACTGCTGGGAAGAAGCAATGGA |
| | | AAAAATGATTGAAAACTTAAGACAAACAGAACTGAAACTGACT |
| | | CGGAAGGCAGCCTATGTGAGATACTTCAATAGCTCAGCCTTC |
| | | TTCTTCTCAGGGTTCTTTGTGGTGTTTTTATCTGTGCTTCCCT |
| | | ATGCACTAATCAAAGGAATCATCCTCCGGAAAATATTCACCA |
| | | CCATCTCATTCTGCATTGTTCTGCGCATGGCGGTCACTCGGC |
| | | AATTTCCCTGGGCTGTACAAACATGGTATGACTCTCTTGGAG |
| | | CAATAAACAAAATACAGGATTTCTTACAAAAGCAAGAATATAA |
| | | GACATTGGAATATAACTTAACGACTACAGAAGTAGTGATGGA |
| | | GAATGTAACAGCCTTCTGGGAGGAGGGATTTGGGGAATTATT |
| | | TGAGAAAGCAAAACAAAACAATAACAATAGAAAAACTTCTAAT |
| | | GGTGATGACAGCCTCTTCTTCAGTAATTTCTCACTTCTTGGTA |
| | | CTCCTGTCCTGAAAGATATTAATTTCAAGATAGAAAGAGGAC |
| | | AGTTGTTGGCGGTTGCTGGATCCACTGGAGCAGGCAAGACT |
| | | TCACTTCTAATGATGATTATGGGAGAACTGGAGCCTTCAGAG |
| | | GGTAAAATTAAGCACAGTGGAAGAATTTCATTCTGTTCTCAGT |
| | | TTTCCTGGATTATGCCTGGCACCATTAAAGAAAATATCATCTT |
| | | TGGTGTTTCCTATGATGAATATAGATACAGAAGCGTCATCAAA |
| | | GCATGCCAACTAGAAGAGGACATCTCCAAGTTTGCAGAGAAA |
| | | GACAATATAGTTCTTGGAGAAGGTGGAATCACACTGAGTGGA |
| | | GGTCAACGAGCAAGAATTTCTTTAGCAAGAGCAGTATACAAA |
| | | GATGCTGATTTGTATTTATTAGACTCTCCTTTTGGATACCTAG |
| | | ATGTTTTAACAGAAAAAGAAATATTTGAAAGCTGTGTCTGTAA |
| | | ACTGATGGCTAACAAAACTAGGATTTTGGTCACTTCTAAAATG |
| | | GAACATTTAAAGAAAGCTGACAAAATATTAATTTTGCATGAAG |
| | | GTAGCAGCTATTTTTATGGGACATTTTCAGAACTCCAAAATCT |
| | | ACAGCCAGACTTTAGCTCAAAACTCATGGGATGTGATTCTTT |
| | | CGACCAATTTAGTGCAGAAAGAAGAAATTCAATCCTAACTGA |
| | | GACCTTACACCGTTTCTCATTAGAAGGAGATGCTCCTGTCTC |
| | | CTGGACAGAAACAAAAAAACAATCTTTTAAACAGACTGGAGA |
| | | GTTTGGGGAAAAAAGGAAGAATTCTATTCTCAATCCAATCAA |
| | | CTCTACGCTTCAGGCACGAAGGAGGCAGTCTGTCCTGAACC |
| | | TGATGACACACTCAGTTAACCAAGGTCAGAACATTCACCGAA |
| | | AGACAACAGCATCCACACGAAAAGTGTCACTGGCCCCTCAG |
| | | GCAAACTTGACTGAACTGGATATATATTCAAGAAGGTTATCTC |
| | | AAGAAACTGGCTTGGAAATAAGTGAAGAAATTAACGAAGAAG |
| | | ACTTAAAGGAGTGCCTTTTTGATGATATGGAGAGCATACCAG |
| | | CAGTGACTACATGGAACACATACCTTCGATATATTACTGTCCA |
| | | CAAGAGCTTAATTTTTGTGCTAATTTGGTGCTTAGTAATTTTT |
| | | CTGGCAGAGGTGGCTGCTTCTTTGGTTGTGCTGTGGCTCCTT |
| | | GGAAACACTCCTCTTCAAGACAAAGGGAATAGTACTCATAGT |
| | | AGAAATAACAGCTATGCAGTGATTATCACCAGCACCAGTTCG |
| | | TATTATGTGTTTTACATTTACGTGGGAGTAGCCGACACTTTGC |
| | | TTGCTATGGGATTCTTCAGAGGTCTACCACTGGTGCATACTC |
| | | TAATCACAGTGTCGAAAATTTTACACCACAAAATGTTACATTC |
| | | TGTTCTTCAAGCACCTATGTCAACCCTCAACACGTTGAAAGC |
| | | AGGTGGGATTCTTAATAGATTCTCCAAAGATATAGCAATTTTG |
| | | GATGACCTTCTGCCTCTTACCATATTTGACTTCATCCAGTTGT |
| | | TATTAATTGTGATTGGAGCTATAGCAGTTGTCGCAGTTTTACA |
| | | ACCCTACATCTTTGTTGCAACAGTGCCAGTGATAGTGGCTTT |
| | | TATTATGTTGAGAGCATATTTCCTCCAAACCTCACAGCAACTC |
| | | AAACAACTGGAATCTGAAGGCAGGAGTCCAATTTTCACTCAT |
| | | CTTGTTACAAGCTTAAAAGGACTATGGACACTTCGTGCCTTC |
| | | GGACGGCAGCCTTACTTTGAAACTCTGTTCCACAAAGCTCTG |
| | | AATTTACATACTGCCAACTGGTTCTTGTACCTGTCAACACTGC |
| | | GCTGGTTCCAAATGAGAATAGAAATGATTTTTGTCATCTTCTT |
| | | CATTGCTGTTACCTTCATTTCCATTTTAACAACAGGAGAAGGA |
| | | GAAGGAAGAGTTGGTATTATCCTGACTTTAGCCATGAATATC |
| | | ATGAGTACATTGCAGTGGGCTGTAAACTCCAGCATAGATGTG |
| | | GATAGCTTGATGCGATCTGTGAGCCGAGTCTTTAAGTTCATT |
| | | GACATGCCAACAGAAGGTAAACCTACCAAGTCAACCAAACCA |
| | | TACAAGAATGGCCAACTCTCGAAAGTTATGATTATTGAGAATT |
| | | CACACGTGAAGAAAGATGACATCTGGCCCTCAGGGGGCCAA |
| | | ATGACTGTCAAAGATCTCACAGCAAAATACACAGAAGGTGGA |
| | | AATGCCATATTAGAGAACATTTCCTTCTCAATAAGTCCTGGCC |
| | | AGAGGGTGGGCCTCTTGGGAAGAACTGGATCAGGGAAGAGT |
| | | ACTTTGTTATCAGCTTTTTTGAGACTACTGAACACTGAAGGAG |
| | | AAATCCAGATCGATGGTGTGTCTTGGGATTCAATAACTTTGC |
| | | AACAGTGGAGGAAAGCCTTTGGAGTGATACCACAGAAAGTAT |
| | | TTATTTTTTCTGGAACATTTAGAAAAAACTTGGATCCCTATGA |
| | | ACAGTGGAGTGATCAAGAAATATGGAAAGTTGCAGATGAGGT |
| | | TGGGCTCAGATCTGTGATAGAACAGTTTCCTGGGAAGCTTGA |
| | | CTTTGTCCTTGTGGATGGGGGCTGTGTCCTAAGCCATGGCC |
| | | ACAAGCAGTTGATGTGCTTGGCTAGATCTGTTCTCAGTAAGG |
| | | CGAAGATCTTGCTGCTTGATGAACCCAGTGCTCATTTGGATC |
| | | CAGTAACATACCAAATAATTAGAAGAACTCTAAAACAAGCATT |
| | | TGCTGATTGCACAGTAATTCTCTGTGAACACAGGATAGAAGC |
| | | AATGCTGGAATGCCAACAATTTTTGGTCATAGAAGAGAACAA |
| | | AGTGCGGCAGTACGATTCCATCCAGAAACTGCTGAACGAGA |
| | | GGAGCCTCTTCCGGCAAGCCATCAGCCCCTCCGACAGGGTG |
| | | AAGCTCTTTCCCCACCGGAACTCAAGCAAGTGCAAGTCTAAG |
| | | CCCCAGATTGCTGCTCTGAAAGAGGAGACAGAAGAAGAGGT |
| | | GCAAGATACAAGGCTTTAG |
| 8 | F5 | GAATTCGTGGTGAGCGTCTGGGCATGTCTGGGCATGTCTGG |
| | Enhancer, | GCATGTCTGGGCATGTCGGGCATTCTGGGCGTCTGGGCATG |
| | Tg83 | TCTGGGCATGTCTGGGCATCTCGAGAACGGTGACGTGCACG |
| | Promoter, | CGTGGGCGGAGCCATCACGCAGGTTGCTATATAAGCAGAGC |
| | 5′-UTR, | TCGTTTAGTGAACCGTCAGAGTCGAGCCCGAGAGACCATGC |
| | hCFTRΔR, | AGAGGTCGCCTCTGGAAAAGGCCAGCGTTGTCTCCAAACTTT |
| | 3′- | TTTTCAGCTGGACCAGACCAATTTTGAGGAAAGGATACAGAC |
| | UTR | AGCGCCTGGAATTGTCAGACATATACCAAATCCCTTCTGTTG |
| | | ATTCTGCTGACAATCTATCTGAAAAATTGGAAAGAGAATGGG |
| | | ATAGAGAGCTGGCTTCAAAGAAAAATCCTAAACTCATTAATG |
| | | CCCTTCGGCGATGTTTTTTCTGGAGATTTATGTTCTATGGAAT |
| | | CTTTTTATATTTAGGGGAAGTCACCAAAGCAGTACAGCCTCT |
| | | CTTACTGGGAAGAATCATAGCTTCCTATGACCCGGATAACAA |
| | | GGAGGAACGCTCTATCGCGATTTATCTAGGCATAGGCTTATG |
| | | CCTTCTCTTTATTGTGAGGACACTGCTCCTACACCCAGCCAT |
| | | TTTTGGCCTTCATCACATTGGAATGCAGATGAGAATAGCTAT |
| | | GTTTAGTTTGATTTATAAGAAGACTTTAAAGCTGTCAAGCCGT |
| | | GTTCTAGATAAAATAAGTATTGGACAACTTGTTAGTCTCCTTT |
| | | CCAACAACCTGAACAAATTTGATGAAGGACTTGCATTGGCAC |
| | | ATTTCGTGTGGATCGCTCCTTTGCAAGTGGCACTCCTCATGG |
| | | GGCTAATCTGGGAGTTGTTACAGGCGTCTGCCTTCTGTGGA |
| | | CTTGGTTTCCTGATAGTCCTTGCCCTTTTTCAGGCTGGGCTA |
| | | GGGAGAATGATGATGAAGTACAGAGATCAGAGAGCTGGGAA |
| | | GATCAGTGAAAGACTTGTGATTACCTCAGAAATGATCGAGAA |
| | | CATCCAATCTGTTAAGGCATACTGCTGGGAAGAAGCAATGGA |
| | | AAAAATGATTGAAAACTTAAGACAAACAGAACTGAAACTGACT |
| | | CGGAAGGCAGCCTATGTGAGATACTTCAATAGCTCAGCCTTC |
| | | TTCTTCTCAGGGTTCTTTGTGGTGTTTTTATCTGTGCTTCCCT |
| | | ATGCACTAATCAAAGGAATCATCCTCCGGAAAATATTCACCA |
| | | CCATCTCATTCTGCATTGTTCTGCGCATGGCGGTCACTCGGC |
| | | AATTTCCCTGGGCTGTACAAACATGGTATGACTCTCTTGGAG |
| | | CAATAAACAAAATACAGGATTTCTTACAAAAGCAAGAATATAA |
| | | GACATTGGAATATAACTTAACGACTACAGAAGTAGTGATGGA |
| | | GAATGTAACAGCCTTCTGGGAGGAGGGATTTGGGGAATTATT |
| | | TGAGAAAGCAAAACAAAACAATAACAATAGAAAAACTTCTAAT |
| | | GGTGATGACAGCCTCTTCTTCAGTAATTTCTCACTTCTTGGTA |
| | | CTCCTGTCCTGAAAGATATTAATTTCAAGATAGAAAGAGGAC |
| | | AGTTGTTGGCGGTTGCTGGATCCACTGGAGCAGGCAAGACT |
| | | TCACTTCTAATGATGATTATGGGAGAACTGGAGCCTTCAGAG |
| | | GGTAAAATTAAGCACAGTGGAAGAATTTCATTCTGTTCTCAGT |
| | | TTTCCTGGATTATGCCTGGCACCATTAAAGAAAATATCATCTT |
| | | TGGTGTTTCCTATGATGAATATAGATACAGAAGCGTCATCAAA |
| | | GCATGCCAACTAGAAGAGGACATCTCCAAGTTTGCAGAGAAA |
| | | GACAATATAGTTCTTGGAGAAGGTGGAATCACACTGAGTGGA |
| | | GGTCAACGAGCAAGAATTTCTTTAGCAAGAGCAGTATACAAA |
| | | GATGCTGATTTGTATTTATTAGACTCTCCTTTTGGATACCTAG |
| | | ATGTTTTAACAGAAAAAGAAATATTTGAAAGCTGTGTCTGTAA |
| | | ACTGATGGCTAACAAAACTAGGATTTTGGTCACTTCTAAAATG |
| | | GAACATTTAAAGAAAGCTGACAAAATATTAATTTTGCATGAAG |
| | | GTAGCAGCTATTTTTATGGGACATTTTCAGAACTCCAAAATCT |
| | | ACAGCCAGACTTTAGCTCAAAACTCATGGGATGTGATTCTTT |
| | | CGACCAATTTAGTGCAGAAAGAAGAAATTCAATCCTAACTGA |
| | | GACCTTACACCGTTTCTCATTAGAAGGAGATGCTCCTGTCTC |
| | | CTGGACAGAAACAAAAAAACAATCTTTTAAACAGACTGGAGA |
| | | GTTTGGGGAAAAAAGGAAGAATTCTATTCTCAATCCAATCAA |
| | | CTCTACGCTTCAGGCACGAAGGAGGCAGTCTGTCCTGAACC |
| | | TGATGACACACTCAGTTAACCAAGGTCAGAACATTCACCGAA |
| | | AGACAACAGCATCCACACGAAAAGTGTCACTGGCCCCTCAG |
| | | GCAAACTTGACTGAACTGGATATATATTCAAGAAGGTTATCTC |
| | | AAGAAACTGGCTTGGAAATAAGTGAAGAAATTAACGAAGAAG |
| | | ACTTAAAGGAGTGCCTTTTTGATGATATGGAGAGCATACCAG |
| | | CAGTGACTACATGGAACACATACCTTCGATATATTACTGTCCA |
| | | CAAGAGCTTAATTTTTGTGCTAATTTGGTGCTTAGTAATTTTT |
| | | CTGGCAGAGGTGGCTGCTTCTTTGGTTGTGCTGTGGCTCCTT |
| | | GGAAACACTCCTCTTCAAGACAAAGGGAATAGTACTCATAGT |
| | | AGAAATAACAGCTATGCAGTGATTATCACCAGCACCAGTTCG |
| | | TATTATGTGTTTTACATTTACGTGGGAGTAGCCGACACTTTGC |
| | | TTGCTATGGGATTCTTCAGAGGTCTACCACTGGTGCATACTC |
| | | TAATCACAGTGTCGAAAATTTTACACCACAAAATGTTACATTC |
| | | TGTTCTTCAAGCACCTATGTCAACCCTCAACACGTTGAAAGC |
| | | AGGTGGGATTCTTAATAGATTCTCCAAAGATATAGCAATTTTG |
| | | GATGACCTTCTGCCTCTTACCATATTTGACTTCATCCAGTTGT |
| | | TATTAATTGTGATTGGAGCTATAGCAGTTGTCGCAGTTTTACA |
| | | ACCCTACATCTTTGTTGCAACAGTGCCAGTGATAGTGGCTTT |
| | | TATTATGTTGAGAGCATATTTCCTCCAAACCTCACAGCAACTC |
| | | AAACAACTGGAATCTGAAGGCAGGAGTCCAATTTTCACTCAT |
| | | CTTGTTACAAGCTTAAAAGGACTATGGACACTTCGTGCCTTC |
| | | GGACGGCAGCCTTACTTTGAAACTCTGTTCCACAAAGCTCTG |
| | | AATTTACATACTGCCAACTGGTTCTTGTACCTGTCAACACTGC |
| | | GCTGGTTCCAAATGAGAATAGAAATGATTTTTGTCATCTTCTT |
| | | CATTGCTGTTACCTTCATTTCCATTTTAACAACAGGAGAAGGA |
| | | GAAGGAAGAGTTGGTATTATCCTGACTTTAGCCATGAATATC |
| | | ATGAGTACATTGCAGTGGGCTGTAAACTCCAGCATAGATGTG |
| | | GATAGCTTGATGCGATCTGTGAGCCGAGTCTTTAAGTTCATT |
| | | GACATGCCAACAGAAGGTAAACCTACCAAGTCAACCAAACCA |
| | | TACAAGAATGGCCAACTCTCGAAAGTTATGATTATTGAGAATT |
| | | CACACGTGAAGAAAGATGACATCTGGCCCTCAGGGGGCCAA |
| | | ATGACTGTCAAAGATCTCACAGCAAAATACACAGAAGGTGGA |
| | | AATGCCATATTAGAGAACATTTCCTTCTCAATAAGTCCTGGCC |
| | | AGAGGGTGGGCCTCTTGGGAAGAACTGGATCAGGGAAGAGT |
| | | ACTTTGTTATCAGCTTTTTTGAGACTACTGAACACTGAAGGAG |
| | | AAATCCAGATCGATGGTGTGTCTTGGGATTCAATAACTTTGC |
| | | AACAGTGGAGGAAAGCCTTTGGAGTGATACCACAGAAAGTAT |
| | | TTATTTTTTCTGGAACATTTAGAAAAAACTTGGATCCCTATGA |
| | | ACAGTGGAGTGATCAAGAAATATGGAAAGTTGCAGATGAGGT |
| | | TGGGCTCAGATCTGTGATAGAACAGTTTCCTGGGAAGCTTGA |
| | | CTTTGTCCTTGTGGATGGGGGCTGTGTCCTAAGCCATGGCC |
| | | ACAAGCAGTTGATGTGCTTGGCTAGATCTGTTCTCAGTAAGG |
| | | CGAAGATCTTGCTGCTTGATGAACCCAGTGCTCATTTGGATC |
| | | CAGTAACATACCAAATAATTAGAAGAACTCTAAAACAAGCATT |
| | | TGCTGATTGCACAGTAATTCTCTGTGAACACAGGATAGAAGC |
| | | AATGCTGGAATGCCAACAATTTTTGGTCATAGAAGAGAACAA |
| | | AGTGCGGCAGTACGATTCCATCCAGAAACTGCTGAACGAGA |
| | | GGAGCCTCTTCCGGCAAGCCATCAGCCCCTCCGACAGGGTG |
| | | AAGCTCTTTCCCCACCGGAACTCAAGCAAGTGCAAGTCTAAG |
| | | CCCCAGATTGCTGCTCTGAAAGAGGAGACAGAAGAAGAGGT |
| | | GCAAGATACAAGGCTTTAGAGAGCAGCATAAATGTTGACATG |
| | | GGACATTTGCTCATGGAATTGG |
| 9 | 5′ AAV | TTGGCCACTCCCTCTCTGCGCGCTCGCTCGCTCACTGAGGC |
| | ITR | CGGGCGACCAAAGGTCGCCCGACGCCCGGGCTTTGCCCGG |
| | | GCGGCCTCAGTGAGCGAGCGAGCGCGCAGAGAGGGAGTGG |
| | | CCAACTCCATCACTAGGGGTTCCT |
| 10 | 3′ AAV | AGGAACCCCTAGTGATGGAGTTGGCCACTCCCTCTCTGCGC |
| | ITR | GCTCGCTCGCTCACTGAGGCCGCCCGGGCAAAGCCCGGGC |
| | | GTCGGGCGACCTTTGGTCGCCCGGCCTCAGTGAGCGAGCG |
| | | AGCGCGCAGAGAGGGAGTGGCCAA |
| 11 | 5′ AAV | TTGGCCACTCCCTCTCTGCGCGCTCGCTCGCTCACTGAGGC |
| | ITR | CGGGCGACCAAAGGTCGCCCGACGCCCGGGCTTTGCCCGG |
| | through | GCGGCCTCAGTGAGCGAGCGAGCGCGCAGAGAGGGAGTGG |
| | 3′ AAV | CCAACTCCATCACTAGGGGTTCCTCAGATCTGAATTCGTGGT |
| | ITR | GAGCGTCTGGGCATGTCTGGGCATGTCTGGGCATGTCTGGG |
| | | CATGTCGGGCATTCTGGGCGTCTGGGCATGTCTGGGCATGT |
| | | CTGGGCATCTCGAGAACGGTGACGTGCACGCGTGGGCGGA |
| | | GCCATCACGCAGGTTGCTATATAAGCAGAGCTCGTTTAGTGA |
| | | ACCGTCAGAGTCGAGCCCGAGAGACCATGCAGAGGTCGCCT |
| | | CTGGAAAAGGCCAGCGTTGTCTCCAAACTTTTTTTCAGCTGG |
| | | ACCAGACCAATTTTGAGGAAAGGATACAGACAGCGCCTGGA |
| | | ATTGTCAGACATATACCAAATCCCTTCTGTTGATTCTGCTGAC |
| | | AATCTATCTGAAAAATTGGAAAGAGAATGGGATAGAGAGCTG |
| | | GCTTCAAAGAAAAATCCTAAACTCATTAATGCCCTTCGGCGA |
| | | TGTTTTTTCTGGAGATTTATGTTCTATGGAATCTTTTTATATTT |
| | | AGGGGAAGTCACCAAAGCAGTACAGCCTCTCTTACTGGGAA |
| | | GAATCATAGCTTCCTATGACCCGGATAACAAGGAGGAACGCT |
| | | CTATCGCGATTTATCTAGGCATAGGCTTATGCCTTCTCTTTAT |
| | | TGTGAGGACACTGCTCCTACACCCAGCCATTTTTGGCCTTCA |
| | | TCACATTGGAATGCAGATGAGAATAGCTATGTTTAGTTTGATT |
| | | TATAAGAAGACTTTAAAGCTGTCAAGCCGTGTTCTAGATAAAA |
| | | TAAGTATTGGACAACTTGTTAGTCTCCTTTCCAACAACCTGAA |
| | | CAAATTTGATGAAGGACTTGCATTGGCACATTTCGTGTGGAT |
| | | CGCTCCTTTGCAAGTGGCACTCCTCATGGGGCTAATCTGGG |
| | | AGTTGTTACAGGCGTCTGCCTTCTGTGGACTTGGTTTCCTGA |
| | | TAGTCCTTGCCCTTTTTCAGGCTGGGCTAGGGAGAATGATGA |
| | | TGAAGTACAGAGATCAGAGAGCTGGGAAGATCAGTGAAAGA |
| | | CTTGTGATTACCTCAGAAATGATCGAGAACATCCAATCTGTTA |
| | | AGGCATACTGCTGGGAAGAAGCAATGGAAAAAATGATTGAAA |
| | | ACTTAAGACAAACAGAACTGAAACTGACTCGGAAGGCAGCCT |
| | | ATGTGAGATACTTCAATAGCTCAGCCTTCTTCTTCTCAGGGTT |
| | | CTTTGTGGTGTTTTTATCTGTGCTTCCCTATGCACTAATCAAA |
| | | GGAATCATCCTCCGGAAAATATTCACCACCATCTCATTCTGC |
| | | ATTGTTCTGCGCATGGCGGTCACTCGGCAATTTCCCTGGGCT |
| | | GTACAAACATGGTATGACTCTCTTGGAGCAATAAACAAAATA |
| | | CAGGATTTCTTACAAAAGCAAGAATATAAGACATTGGAATATA |
| | | ACTTAACGACTACAGAAGTAGTGATGGAGAATGTAACAGCCT |
| | | TCTGGGAGGAGGGATTTGGGGAATTATTTGAGAAAGCAAAA |
| | | CAAAACAATAACAATAGAAAAACTTCTAATGGTGATGACAGC |
| | | CTCTTCTTCAGTAATTTCTCACTTCTTGGTACTCCTGTCCTGA |
| | | AAGATATTAATTTCAAGATAGAAAGAGGACAGTTGTTGGCGG |
| | | TTGCTGGATCCACTGGAGCAGGCAAGACTTCACTTCTAATGA |
| | | TGATTATGGGAGAACTGGAGCCTTCAGAGGGTAAAATTAAGC |
| | | ACAGTGGAAGAATTTCATTCTGTTCTCAGTTTTCCTGGATTAT |
| | | GCCTGGCACCATTAAAGAAAATATCATCTTTGGTGTTTCCTAT |
| | | GATGAATATAGATACAGAAGCGTCATCAAAGCATGCCAACTA |
| | | GAAGAGGACATCTCCAAGTTTGCAGAGAAAGACAATATAGTT |
| | | CTTGGAGAAGGTGGAATCACACTGAGTGGAGGTCAACGAGC |
| | | AAGAATTTCTTTAGCAAGAGCAGTATACAAAGATGCTGATTTG |
| | | TATTTATTAGACTCTCCTTTTGGATACCTAGATGTTTTAACAG |
| | | AAAAAGAAATATTTGAAAGCTGTGTCTGTAAACTGATGGCTAA |
| | | CAAAACTAGGATTTTGGTCACTTCTAAAATGGAACATTTAAAG |
| | | AAAGCTGACAAAATATTAATTTTGCATGAAGGTAGCAGCTATT |
| | | TTTATGGGACATTTTCAGAACTCCAAAATCTACAGCCAGACTT |
| | | TAGCTCAAAACTCATGGGATGTGATTCTTTCGACCAATTTAGT |
| | | GCAGAAAGAAGAAATTCAATCCTAACTGAGACCTTACACCGT |
| | | TTCTCATTAGAAGGAGATGCTCCTGTCTCCTGGACAGAAACA |
| | | AAAAAACAATCTTTTAAACAGACTGGAGAGTTTGGGGAAAAA |
| | | AGGAAGAATTCTATTCTCAATCCAATCAACTCTACGCTTCAGG |
| | | CACGAAGGAGGCAGTCTGTCCTGAACCTGATGACACACTCA |
| | | GTTAACCAAGGTCAGAACATTCACCGAAAGACAACAGCATCC |
| | | ACACGAAAAGTGTCACTGGCCCCTCAGGCAAACTTGACTGA |
| | | ACTGGATATATATTCAAGAAGGTTATCTCAAGAAACTGGCTTG |
| | | GAAATAAGTGAAGAAATTAACGAAGAAGACTTAAAGGAGTGC |
| | | CTTTTTGATGATATGGAGAGCATACCAGCAGTGACTACATGG |
| | | AACACATACCTTCGATATATTACTGTCCACAAGAGCTTAATTT |
| | | TTGTGCTAATTTGGTGCTTAGTAATTTTTCTGGCAGAGGTGG |
| | | CTGCTTCTTTGGTTGTGCTGTGGCTCCTTGGAAACACTCCTC |
| | | TTCAAGACAAAGGGAATAGTACTCATAGTAGAAATAACAGCT |
| | | ATGCAGTGATTATCACCAGCACCAGTTCGTATTATGTGTTTTA |
| | | CATTTACGTGGGAGTAGCCGACACTTTGCTTGCTATGGGATT |
| | | CTTCAGAGGTCTACCACTGGTGCATACTCTAATCACAGTGTC |
| | | GAAAATTTTACACCACAAAATGTTACATTCTGTTCTTCAAGCA |
| | | CCTATGTCAACCCTCAACACGTTGAAAGCAGGTGGGATTCTT |
| | | AATAGATTCTCCAAAGATATAGCAATTTTGGATGACCTTCTGC |
| | | CTCTTACCATATTTGACTTCATCCAGTTGTTATTAATTGTGATT |
| | | GGAGCTATAGCAGTTGTCGCAGTTTTACAACCCTACATCTTT |
| | | GTTGCAACAGTGCCAGTGATAGTGGCTTTTATTATGTTGAGA |
| | | GCATATTTCCTCCAAACCTCACAGCAACTCAAACAACTGGAA |
| | | TCTGAAGGCAGGAGTCCAATTTTCACTCATCTTGTTACAAGC |
| | | TTAAAAGGACTATGGACACTTCGTGCCTTCGGACGGCAGCCT |
| | | TACTTTGAAACTCTGTTCCACAAAGCTCTGAATTTACATACTG |
| | | CCAACTGGTTCTTGTACCTGTCAACACTGCGCTGGTTCCAAA |
| | | TGAGAATAGAAATGATTTTTGTCATCTTCTTCATTGCTGTTAC |
| | | CTTCATTTCCATTTTAACAACAGGAGAAGGAGAAGGAAGAGT |
| | | TGGTATTATCCTGACTTTAGCCATGAATATCATGAGTACATTG |
| | | CAGTGGGCTGTAAACTCCAGCATAGATGTGGATAGCTTGATG |
| | | CGATCTGTGAGCCGAGTCTTTAAGTTCATTGACATGCCAACA |
| | | GAAGGTAAACCTACCAAGTCAACCAAACCATACAAGAATGGC |
| | | CAACTCTCGAAAGTTATGATTATTGAGAATTCACACGTGAAGA |
| | | AAGATGACATCTGGCCCTCAGGGGGCCAAATGACTGTCAAA |
| | | GATCTCACAGCAAAATACACAGAAGGTGGAAATGCCATATTA |
| | | GAGAACATTTCCTTCTCAATAAGTCCTGGCCAGAGGGTGGG |
| | | CCTCTTGGGAAGAACTGGATCAGGGAAGAGTACTTTGTTATC |
| | | AGCTTTTTTGAGACTACTGAACACTGAAGGAGAAATCCAGAT |
| | | CGATGGTGTGTCTTGGGATTCAATAACTTTGCAACAGTGGAG |
| | | GAAAGCCTTTGGAGTGATACCACAGAAAGTATTTATTTTTTCT |
| | | GGAACATTTAGAAAAAACTTGGATCCCTATGAACAGTGGAGT |
| | | GATCAAGAAATATGGAAAGTTGCAGATGAGGTTGGGCTCAG |
| | | ATCTGTGATAGAACAGTTTCCTGGGAAGCTTGACTTTGTCCT |
| | | TGTGGATGGGGGCTGTGTCCTAAGCCATGGCCACAAGCAGT |
| | | TGATGTGCTTGGCTAGATCTGTTCTCAGTAAGGCGAAGATCT |
| | | TGCTGCTTGATGAACCCAGTGCTCATTTGGATCCAGTAACAT |
| | | ACCAAATAATTAGAAGAACTCTAAAACAAGCATTTGCTGATTG |
| | | CACAGTAATTCTCTGTGAACACAGGATAGAAGCAATGCTGGA |
| | | ATGCCAACAATTTTTGGTCATAGAAGAGAACAAAGTGCGGCA |
| | | GTACGATTCCATCCAGAAACTGCTGAACGAGAGGAGCCTCTT |
| | | CCGGCAAGCCATCAGCCCCTCCGACAGGGTGAAGCTCTTTC |
| | | CCCACCGGAACTCAAGCAAGTGCAAGTCTAAGCCCCAGATT |
| | | GCTGCTCTGAAAGAGGAGACAGAAGAAGAGGTGCAAGATAC |
| | | AAGGCTTTAGAGAGCAGCATAAATGTTGACATGGGACATTTG |
| | | CTCATGGAATTGGCAGGCCTAATAAAGAGCTCAGATGCATCG |
| | | ATCAGAGTGTGTTGGTTTTTTGTGTGTACTGAGGAACCCCTA |
| | | GTGATGGAGTTGGCCACTCCCTCTCTGCGCGCTCGCTCGCT |
| | | CACTGAGGCCGCCCGGGCAAAGCCCGGGCGTCGGGCGACC |
| | | TTTGGTCGCCCGGCCTCAGTGAGCGAGCGAGCGCGCAGAG |
| | | AGGGAGTGGCCAA |
| 12 | pAV- | TTGGCCACTCCCTCTCTGCGCGCTCGCTCGCTCACTGAGGC |
| | F5tg83- | CGGGCGACCAAAGGTCGCCCGACGCCCGGGCTTTGCCCGG |
| | hCFTR- | GCGGCCTCAGTGAGCGAGCGAGCGCGCAGAGAGGGAGTGG |
| | dR | CCAACTCCATCACTAGGGGTTCCTCAGATCTGAATTCGTGGT |
| | vector | GAGCGTCTGGGCATGTCTGGGCATGTCTGGGCATGTCTGGG |
| | | CATGTCGGGCATTCTGGGCGTCTGGGCATGTCTGGGCATGT |
| | | CTGGGCATCTCGAGAACGGTGACGTGCACGCGTGGGCGGA |
| | | GCCATCACGCAGGTTGCTATATAAGCAGAGCTCGTTTAGTGA |
| | | ACCGTCAGAGTCGAGCCCGAGAGACCATGCAGAGGTCGCCT |
| | | CTGGAAAAGGCCAGCGTTGTCTCCAAACTTTTTTTCAGCTGG |
| | | ACCAGACCAATTTTGAGGAAAGGATACAGACAGCGCCTGGA |
| | | ATTGTCAGACATATACCAAATCCCTTCTGTTGATTCTGCTGAC |
| | | AATCTATCTGAAAAATTGGAAAGAGAATGGGATAGAGAGCTG |
| | | GCTTCAAAGAAAAATCCTAAACTCATTAATGCCCTTCGGCGA |
| | | TGTTTTTTCTGGAGATTTATGTTCTATGGAATCTTTTTATATTT |
| | | AGGGGAAGTCACCAAAGCAGTACAGCCTCTCTTACTGGGAA |
| | | GAATCATAGCTTCCTATGACCCGGATAACAAGGAGGAACGCT |
| | | CTATCGCGATTTATCTAGGCATAGGCTTATGCCTTCTCTTTAT |
| | | TGTGAGGACACTGCTCCTACACCCAGCCATTTTTGGCCTTCA |
| | | TCACATTGGAATGCAGATGAGAATAGCTATGTTTAGTTTGATT |
| | | TATAAGAAGACTTTAAAGCTGTCAAGCCGTGTTCTAGATAAAA |
| | | TAAGTATTGGACAACTTGTTAGTCTCCTTTCCAACAACCTGAA |
| | | CAAATTTGATGAAGGACTTGCATTGGCACATTTCGTGTGGAT |
| | | CGCTCCTTTGCAAGTGGCACTCCTCATGGGGCTAATCTGGG |
| | | AGTTGTTACAGGCGTCTGCCTTCTGTGGACTTGGTTTCCTGA |
| | | TAGTCCTTGCCCTTTTTCAGGCTGGGCTAGGGAGAATGATGA |
| | | TGAAGTACAGAGATCAGAGAGCTGGGAAGATCAGTGAAAGA |
| | | CTTGTGATTACCTCAGAAATGATCGAGAACATCCAATCTGTTA |
| | | AGGCATACTGCTGGGAAGAAGCAATGGAAAAAATGATTGAAA |
| | | ACTTAAGACAAACAGAACTGAAACTGACTCGGAAGGCAGCCT |
| | | ATGTGAGATACTTCAATAGCTCAGCCTTCTTCTTCTCAGGGTT |
| | | CTTTGTGGTGTTTTTATCTGTGCTTCCCTATGCACTAATCAAA |
| | | GGAATCATCCTCCGGAAAATATTCACCACCATCTCATTCTGC |
| | | ATTGTTCTGCGCATGGCGGTCACTCGGCAATTTCCCTGGGCT |
| | | GTACAAACATGGTATGACTCTCTTGGAGCAATAAACAAAATA |
| | | CAGGATTTCTTACAAAAGCAAGAATATAAGACATTGGAATATA |
| | | ACTTAACGACTACAGAAGTAGTGATGGAGAATGTAACAGCCT |
| | | TCTGGGAGGAGGGATTTGGGGAATTATTTGAGAAAGCAAAA |
| | | CAAAACAATAACAATAGAAAAACTTCTAATGGTGATGACAGC |
| | | CTCTTCTTCAGTAATTTCTCACTTCTTGGTACTCCTGTCCTGA |
| | | AAGATATTAATTTCAAGATAGAAAGAGGACAGTTGTTGGCGG |
| | | TTGCTGGATCCACTGGAGCAGGCAAGACTTCACTTCTAATGA |
| | | TGATTATGGGAGAACTGGAGCCTTCAGAGGGTAAAATTAAGC |
| | | ACAGTGGAAGAATTTCATTCTGTTCTCAGTTTTCCTGGATTAT |
| | | GCCTGGCACCATTAAAGAAAATATCATCTTTGGTGTTTCCTAT |
| | | GATGAATATAGATACAGAAGCGTCATCAAAGCATGCCAACTA |
| | | GAAGAGGACATCTCCAAGTTTGCAGAGAAAGACAATATAGTT |
| | | CTTGGAGAAGGTGGAATCACACTGAGTGGAGGTCAACGAGC |
| | | AAGAATTTCTTTAGCAAGAGCAGTATACAAAGATGCTGATTTG |
| | | TATTTATTAGACTCTCCTTTTGGATACCTAGATGTTTTAACAG |
| | | AAAAAGAAATATTTGAAAGCTGTGTCTGTAAACTGATGGCTAA |
| | | CAAAACTAGGATTTTGGTCACTTCTAAAATGGAACATTTAAAG |
| | | AAAGCTGACAAAATATTAATTTTGCATGAAGGTAGCAGCTATT |
| | | TTTATGGGACATTTTCAGAACTCCAAAATCTACAGCCAGACTT |
| | | TAGCTCAAAACTCATGGGATGTGATTCTTTCGACCAATTTAGT |
| | | GCAGAAAGAAGAAATTCAATCCTAACTGAGACCTTACACCGT |
| | | TTCTCATTAGAAGGAGATGCTCCTGTCTCCTGGACAGAAACA |
| | | AAAAAACAATCTTTTAAACAGACTGGAGAGTTTGGGGAAAAA |
| | | AGGAAGAATTCTATTCTCAATCCAATCAACTCTACGCTTCAGG |
| | | CACGAAGGAGGCAGTCTGTCCTGAACCTGATGACACACTCA |
| | | GTTAACCAAGGTCAGAACATTCACCGAAAGACAACAGCATCC |
| | | ACACGAAAAGTGTCACTGGCCCCTCAGGCAAACTTGACTGA |
| | | ACTGGATATATATTCAAGAAGGTTATCTCAAGAAACTGGCTTG |
| | | GAAATAAGTGAAGAAATTAACGAAGAAGACTTAAAGGAGTGC |
| | | CTTTTTGATGATATGGAGAGCATACCAGCAGTGACTACATGG |
| | | AACACATACCTTCGATATATTACTGTCCACAAGAGCTTAATTT |
| | | TTGTGCTAATTTGGTGCTTAGTAATTTTTCTGGCAGAGGTGG |
| | | CTGCTTCTTTGGTTGTGCTGTGGCTCCTTGGAAACACTCCTC |
| | | TTCAAGACAAAGGGAATAGTACTCATAGTAGAAATAACAGCT |
| | | ATGCAGTGATTATCACCAGCACCAGTTCGTATTATGTGTTTTA |
| | | CATTTACGTGGGAGTAGCCGACACTTTGCTTGCTATGGGATT |
| | | CTTCAGAGGTCTACCACTGGTGCATACTCTAATCACAGTGTC |
| | | GAAAATTTTACACCACAAAATGTTACATTCTGTTCTTCAAGCA |
| | | CCTATGTCAACCCTCAACACGTTGAAAGCAGGTGGGATTCTT |
| | | AATAGATTCTCCAAAGATATAGCAATTTTGGATGACCTTCTGC |
| | | CTCTTACCATATTTGACTTCATCCAGTTGTTATTAATTGTGATT |
| | | GGAGCTATAGCAGTTGTCGCAGTTTTACAACCCTACATCTTT |
| | | GTTGCAACAGTGCCAGTGATAGTGGCTTTTATTATGTTGAGA |
| | | GCATATTTCCTCCAAACCTCACAGCAACTCAAACAACTGGAA |
| | | TCTGAAGGCAGGAGTCCAATTTTCACTCATCTTGTTACAAGC |
| | | TTAAAAGGACTATGGACACTTCGTGCCTTCGGACGGCAGCCT |
| | | TACTTTGAAACTCTGTTCCACAAAGCTCTGAATTTACATACTG |
| | | CCAACTGGTTCTTGTACCTGTCAACACTGCGCTGGTTCCAAA |
| | | TGAGAATAGAAATGATTTTTGTCATCTTCTTCATTGCTGTTAC |
| | | CTTCATTTCCATTTTAACAACAGGAGAAGGAGAAGGAAGAGT |
| | | TGGTATTATCCTGACTTTAGCCATGAATATCATGAGTACATTG |
| | | CAGTGGGCTGTAAACTCCAGCATAGATGTGGATAGCTTGATG |
| | | CGATCTGTGAGCCGAGTCTTTAAGTTCATTGACATGCCAACA |
| | | GAAGGTAAACCTACCAAGTCAACCAAACCATACAAGAATGGC |
| | | CAACTCTCGAAAGTTATGATTATTGAGAATTCACACGTGAAGA |
| | | AAGATGACATCTGGCCCTCAGGGGGCCAAATGACTGTCAAA |
| | | GATCTCACAGCAAAATACACAGAAGGTGGAAATGCCATATTA |
| | | GAGAACATTTCCTTCTCAATAAGTCCTGGCCAGAGGGTGGG |
| | | CCTCTTGGGAAGAACTGGATCAGGGAAGAGTACTTTGTTATC |
| | | AGCTTTTTTGAGACTACTGAACACTGAAGGAGAAATCCAGAT |
| | | CGATGGTGTGTCTTGGGATTCAATAACTTTGCAACAGTGGAG |
| | | GAAAGCCTTTGGAGTGATACCACAGAAAGTATTTATTTTTTCT |
| | | GGAACATTTAGAAAAAACTTGGATCCCTATGAACAGTGGAGT |
| | | GATCAAGAAATATGGAAAGTTGCAGATGAGGTTGGGCTCAG |
| | | ATCTGTGATAGAACAGTTTCCTGGGAAGCTTGACTTTGTCCT |
| | | TGTGGATGGGGGCTGTGTCCTAAGCCATGGCCACAAGCAGT |
| | | TGATGTGCTTGGCTAGATCTGTTCTCAGTAAGGCGAAGATCT |
| | | TGCTGCTTGATGAACCCAGTGCTCATTTGGATCCAGTAACAT |
| | | ACCAAATAATTAGAAGAACTCTAAAACAAGCATTTGCTGATTG |
| | | CACAGTAATTCTCTGTGAACACAGGATAGAAGCAATGCTGGA |
| | | ATGCCAACAATTTTTGGTCATAGAAGAGAACAAAGTGCGGCA |
| | | GTACGATTCCATCCAGAAACTGCTGAACGAGAGGAGCCTCTT |
| | | CCGGCAAGCCATCAGCCCCTCCGACAGGGTGAAGCTCTTTC |
| | | CCCACCGGAACTCAAGCAAGTGCAAGTCTAAGCCCCAGATT |
| | | GCTGCTCTGAAAGAGGAGACAGAAGAAGAGGTGCAAGATAC |
| | | AAGGCTTTAGAGAGCAGCATAAATGTTGACATGGGACATTTG |
| | | CTCATGGAATTGGCAGGCCTAATAAAGAGCTCAGATGCATCG |
| | | ATCAGAGTGTGTTGGTTTTTTGTGTGTACTGAGGAACCCCTA |
| | | GTGATGGAGTTGGCCACTCCCTCTCTGCGCGCTCGCTCGCT |
| | | CACTGAGGCCGCCCGGGCAAAGCCCGGGCGTCGGGCGACC |
| | | TTTGGTCGCCCGGCCTCAGTGAGCGAGCGAGCGCGCAGAG |
| | | AGGGAGTGGCCAACCCCCCCCCCCCCCCCCCTGCAGCCAG |
| | | CTGGCGTAATAGCGAAGAGGCCCGCACCGATCGCCCTTCCC |
| | | AACAGTTGCGTAGCCTGAATGGCGAATGGCGCGACGCGCCC |
| | | TGTAGCGGCGCATTAAGCGCGGCGGGTGTGGTGGTTACGC |
| | | GCAGCGTGACCGCTACACTTGCCAGCGCCCTAGCGCCCGCT |
| | | CCTTTCGCTTTCTTCCCTTCCTTTCTCGCCACGTTCGCCGGC |
| | | TTTCCCCGTCAAGCTCTAAATCGGGGGCTCCCTTTAGGGTTC |
| | | CGATTTAGTGCTTTACGGCACCTCGACCCCAAAAAACTTGAT |
| | | TAGGGTGATGGTTCACGTAGTGGGCCATCGCCCTGATAGAC |
| | | GGTTTTTCGCCCTTTGACGTTGGAGTCCACGTTCTTTAATAGT |
| | | GGACTCTTGTTCCAAACTGGAACAACACTCAACCCTATCTCG |
| | | GTCTATTCTTTTGATTTATAAGGGATTTTGCCGATTTCGGCCT |
| | | ATTGGTTAAAAAATGAGCTGATTTAACAAAAATTTAACGCGAA |
| | | TTTTAACAAAATATTAACGTTTACAATTTCCTGATGCGGTATTT |
| | | TCTCCTTACGCATCTGTGCGGTATTTCACACCGCATATGGTG |
| | | CACTCTCAGTACAATCTGCTCTGATGCCGCATAGTTAAGCCA |
| | | GCCCCGACACCCGCCAACACCCGCTGACGCGCCCTGACGG |
| | | GCTTGTCTGCTCCCGGCATCCGCTTACAGACAAGCTGTGAC |
| | | CGTCTCCGGGAGCTGCATGTGTCAGAGGTTTTCACCGTCAT |
| | | CACCGAAACGCGCGAGACGAAAGGGCCTCGTGATACGCCTA |
| | | TTTTTATAGGTTAATGTCATGATAATAATGGTTTCTTAGACGT |
| | | CAGGTGGCACTTTTCGGGGAAATGTGCGCGGAACCCCTATT |
| | | TGTTTATTTTTCTAAATACATTCAAATATGTATCCGCTCATGAG |
| | | ACAATAACCCTGATAAATGCTTCAATAATATTGAAAAAGGAAG |
| | | AGTATGAGTATTCAACATTTCCGTGTCGCCCTTATTCCCTTTT |
| | | TTGCGGCATTTTGCCTTCCTGTTTTTGCTCACCCAGAAACGC |
| | | TGGTGAAAGTAAAAGATGCTGAAGATCAGTTGGGTGCACGA |
| | | GTGGGTTACATCGAACTGGATCTCAACAGCGGTAAGATCCTT |
| | | GAGAGTTTTCGCCCCGAAGAACGTTTTCCAATGATGAGCACT |
| | | TTTAAAGTTCTGCTATGTGGCGCGGTATTATCCCGTATTGAC |
| | | GCCGGGCAAGAGCAACTCGGTCGCCGCATACACTATTCTCA |
| | | GAATGACTTGGTTGAGTACTCACCAGTCACAGAAAAGCATCT |
| | | TACGGATGGCATGACAGTAAGAGAATTATGCAGTGCTGCCAT |
| | | AACCATGAGTGATAACACTGCGGCCAACTTACTTCTGACAAC |
| | | GATCGGAGGACCGAAGGAGCTAACCGCTTTTTTGCACAACAT |
| | | GGGGGATCATGTAACTCGCCTTGATCGTTGGGAACCGGAGC |
| | | TGAATGAAGCCATACCAAACGACGAGCGTGACACCACGATG |
| | | CCTGTAGCAATGGCAACAACGTTGCGCAAACTATTAACTGGC |
| | | GAACTACTTACTCTAGCTTCCCGGCAACAATTAATAGACTGG |
| | | ATGGAGGCGGATAAAGTTGCAGGACCACTTCTGCGCTCGGC |
| | | CCTTCCGGCTGGCTGGTTTATTGCTGATAAATCTGGAGCCGG |
| | | TGAGCGTGGGTCTCGCGGTATCATTGCAGCACTGGGGCCAG |
| | | ATGGTAAGCCCTCCCGTATCGTAGTTATCTACACGACGGGGA |
| | | GTCAGGCAACTATGGATGAACGAAATAGACAGATCGCTGAG |
| | | ATAGGTGCCTCACTGATTAAGCATTGGTAACTGTCAGACCAA |
| | | GTTTACTCATATATACTTTAGATTGATTTAAAACTTCATTTTTA |
| | | ATTTAAAAGGATCTAGGTGAAGATCCTTTTTGATAATCTCATG |
| | | ACCAAAATCCCTTAACGTGAGTTTTCGTTCCACTGAGCGTCA |
| | | GACCCCGTAGAAAAGATCAAAGGATCTTCTTGAGATCCTTTT |
| | | TTTCTGCGCGTAATCTGCTGCTTGCAAACAAAAAAACCACCG |
| | | CTACCAGCGGTGGTTTGTTTGCCGGATCAAGAGCTACCAACT |
| | | CTTTTTCCGAAGGTAACTGGCTTCAGCAGAGCGCAGATACCA |
| | | AATACTGTCCTTCTAGTGTAGCCGTAGTTAGGCCACCACTTC |
| | | AAGAACTCTGTAGCACCGCCTACATACCTCGCTCTGCTAATC |
| | | CTGTTACCAGTGGCTGCTGCCAGTGGCGATAAGTCGTGTCTT |
| | | ACCGGGTTGGACTCAAGACGATAGTTACCGGATAAGGCGCA |
| | | GCGGTCGGGCTGAACGGGGGGTTCGTGCACACAGCCCAGC |
| | | TTGGAGCGAACGACCTACACCGAACTGAGATACCTACAGCG |
| | | TGAGCATTGAGAAAGCGCCACGCTTCCCGAAGGGAGAAAGG |
| | | CGGACAGGTATCCGGTAAGCGGCAGGGTCGGAACAGGAGA |
| | | GCGCACGAGGGAGCTTCCAGGGGGAAACGCCTGGTATCTTT |
| | | ATAGTCCTGTCGGGTTTCGCCACCTCTGACTTGAGCGTCGAT |
| | | TTTTGTGATGCTCGTCAGGGGGGCGGAGCCTATGGAAAAAC |
| | | GCCAGCAACGCGGCCTTTTTACGGTTCCTGGCCTTTTGCTG |
| | | GCCTTTTGCTCACATGTTCTTTCCTGCGTTATCCCCTGATTCT |
| | | GTGGATAACCGTATTACCGCCTTTGAGTGAGCTGATACCGCT |
| | | CGCCGCAGCCGAACGACCGAGCGCAGCGAGTCAGTGAGCG |
| | | AGGAAGCGGAAGAGCGCCCAATACGCAAACCGCCTCTCCCC |
| | | GCGCGTTGGCCGATTCATTAATGCAGCTGGGCTGCAGGGGG |
| | | GGGGGGGGGGGGG |
| 13 | AV.TL65 | MAADGYLPDWLEDTLSEGIRQWWKLKPGPPPPKPAERHKDDS |
| | capsid | RGLVLPGYKYLGPFNGLDKGEPVNEADAAALEHDKAYDRQLDS |
| | protein | GDNPYLKYNHADAEFQERLKEDTSFGGNLGRAVFQAKKRVLEP |
| | | FGLVEEGAKTAPTGKRIDDHFPKRKKARTEEDSKPSTSSDAEA |
| | | GPSGSQQLQIPAQPASSLGADTMSAGGGGPLGDNNQGADGV |
| | | GNASGDWHCDSTWMGDRVVTKSTRTWVLPSYNNHQYREIKS |
| | | GSVDGSNANAYFGYSTPWGYFDFNRFHSHWSPRDWQRLINN |
| | | YWGFRPRSLRVKIFNIQVKEVTVQDSTTTIANNLTSTVQVFTDD |
| | | DYQLPYVVGNGTEGCLPAFPPQVFTLPQYGYATLNRDNTENPT |
| | | ERSSFFCLEYFPSKMLRTGNNFEFTYNFEEVPFHSSFAPSQNLF |
| | | KLANPLVDQYLYRFVSTNNTGGVQFNKNLAGRYANTYKNWFP |
| | | GPMGRTQGWNLGSGVNRASVSAFATTNRMELEGASYQVPPQ |
| | | PNGMTNNLQGSNTYALENTMIFNSQPANPGTTATYLEGNMLIT |
| | | SESETQPVNRVAYNVGGQMATNNQSSTTAPTTGTYNLQEIVPG |
| | | SVWMERDVYLQGPIWAKIPETGAHFHPSPAMGGFGLKHPPPM |
| | | MLIKNTPVPGNITSFSDVPVSSFITQYSTGQVTVEMEWELKKEN |
| | | SKRWNPEIQYTNNYNDPQFVDFAPDSTGEYRTTRPIGTRYLTR |
| | | PL |
| 14 | F5 | GTGGTGAGCGTCTGGGCATGTCTGGGCATGTCTGGGCATGT |
| | enhancer | CTGGGCATGTCGGGCATTCTGGGCGTCTGGGCATGTCTGGG |
| | | CATGTCTGGGCAT |
| 15 | 5′ AAV | CCACTCCCTCTCTGCGCGCTCGCTCGCTCACTGAGGCCGCC |
| | ITR (flop) | CGGGCAAAGCCCGGGCGTCGGGCGACCTTTGGTCGCCCGG |
| | | CCTCAGTGAGCGAGCGAGCGCGCAGAGAGGGAGTGGCCAA |
| | | CTCCATCACTAGGGGTTCCT |
| 16 | 3′ AAV | AGGAACCCCTAGTGATGGAGTTGGCCACTCCCTCTCTGCGC |
| | ITR (flop) | GCTCGCTCGCTCACTGAGGCCGGGCGACCAAAGGTCGCCC |
| | | GACGCCCGGGCTTTGCCCGGGCGGCCTCAGTGAGCGAGCG |
| | | AGCGCGCAGAGAGGGAGTGGCC |
| 17 | 5′ AAV | CCACTCCCTCTCTGCGCGCTCGCTCGCTCACTGAGGCCGCC |
| | ITR (flop) | CGGGCAAAGCCCGGGCGTCGGGCGACCTTTGGTCGCCCGG |
| | through | CCTCAGTGAGCGAGCGAGCGCGCAGAGAGGGAGTGGCCAA |
| | 3′ AAV | CTCCATCACTAGGGGTTCCTCAGATCTGAATTCGTGGTGAGC |
| | ITR (flop) | GTCTGGGCATGTCTGGGCATGTCTGGGCATGTCTGGGCATG |
| | | TCGGGCATTCTGGGCGTCTGGGCATGTCTGGGCATGTCTGG |
| | | GCATCTCGAGAACGGTGACGTGCACGCGTGGGCGGAGCCA |
| | | TCACGCAGGTTGCTATATAAGCAGAGCTCGTTTAGTGAACCG |
| | | TCAGAGTCGAGCCCGAGAGACCATGCAGAGGTCGCCTCTGG |
| | | AAAAGGCCAGCGTTGTCTCCAAACTTTTTTTCAGCTGGACCA |
| | | GACCAATTTTGAGGAAAGGATACAGACAGCGCCTGGAATTGT |
| | | CAGACATATACCAAATCCCTTCTGTTGATTCTGCTGACAATCT |
| | | ATCTGAAAAATTGGAAAGAGAATGGGATAGAGAGCTGGCTTC |
| | | AAAGAAAAATCCTAAACTCATTAATGCCCTTCGGCGATGTTTT |
| | | TTCTGGAGATTTATGTTCTATGGAATCTTTTTATATTTAGGGG |
| | | AAGTCACCAAAGCAGTACAGCCTCTCTTACTGGGAAGAATCA |
| | | TAGCTTCCTATGACCCGGATAACAAGGAGGAACGCTCTATCG |
| | | CGATTTATCTAGGCATAGGCTTATGCCTTCTCTTTATTGTGAG |
| | | GACACTGCTCCTACACCCAGCCATTTTTGGCCTTCATCACAT |
| | | TGGAATGCAGATGAGAATAGCTATGTTTAGTTTGATTTATAAG |
| | | AAGACTTTAAAGCTGTCAAGCCGTGTTCTAGATAAAATAAGTA |
| | | TTGGACAACTTGTTAGTCTCCTTTCCAACAACCTGAACAAATT |
| | | TGATGAAGGACTTGCATTGGCACATTTCGTGTGGATCGCTCC |
| | | TTTGCAAGTGGCACTCCTCATGGGGCTAATCTGGGAGTTGTT |
| | | ACAGGCGTCTGCCTTCTGTGGACTTGGTTTCCTGATAGTCCT |
| | | TGCCCTTTTTCAGGCTGGGCTAGGGAGAATGATGATGAAGTA |
| | | CAGAGATCAGAGAGCTGGGAAGATCAGTGAAAGACTTGTGA |
| | | TTACCTCAGAAATGATCGAGAACATCCAATCTGTTAAGGCAT |
| | | ACTGCTGGGAAGAAGCAATGGAAAAAATGATTGAAAACTTAA |
| | | GACAAACAGAACTGAAACTGACTCGGAAGGCAGCCTATGTG |
| | | AGATACTTCAATAGCTCAGCCTTCTTCTTCTCAGGGTTCTTTG |
| | | TGGTGTTTTTATCTGTGCTTCCCTATGCACTAATCAAAGGAAT |
| | | CATCCTCCGGAAAATATTCACCACCATCTCATTCTGCATTGTT |
| | | CTGCGCATGGCGGTCACTCGGCAATTTCCCTGGGCTGTACA |
| | | AACATGGTATGACTCTCTTGGAGCAATAAACAAAATACAGGA |
| | | TTTCTTACAAAAGCAAGAATATAAGACATTGGAATATAACTTA |
| | | ACGACTACAGAAGTAGTGATGGAGAATGTAACAGCCTTCTGG |
| | | GAGGAGGGATTTGGGGAATTATTTGAGAAAGCAAAACAAAAC |
| | | AATAACAATAGAAAAACTTCTAATGGTGATGACAGCCTCTTCT |
| | | TCAGTAATTTCTCACTTCTTGGTACTCCTGTCCTGAAAGATAT |
| | | TAATTTCAAGATAGAAAGAGGACAGTTGTTGGCGGTTGCTGG |
| | | ATCCACTGGAGCAGGCAAGACTTCACTTCTAATGATGATTAT |
| | | GGGAGAACTGGAGCCTTCAGAGGGTAAAATTAAGCACAGTG |
| | | GAAGAATTTCATTCTGTTCTCAGTTTTCCTGGATTATGCCTGG |
| | | CACCATTAAAGAAAATATCATCTTTGGTGTTTCCTATGATGAA |
| | | TATAGATACAGAAGCGTCATCAAAGCATGCCAACTAGAAGAG |
| | | GACATCTCCAAGTTTGCAGAGAAAGACAATATAGTTCTTGGA |
| | | GAAGGTGGAATCACACTGAGTGGAGGTCAACGAGCAAGAAT |
| | | TTCTTTAGCAAGAGCAGTATACAAAGATGCTGATTTGTATTTA |
| | | TTAGACTCTCCTTTTGGATACCTAGATGTTTTAACAGAAAAAG |
| | | AAATATTTGAAAGCTGTGTCTGTAAACTGATGGCTAACAAAAC |
| | | TAGGATTTTGGTCACTTCTAAAATGGAACATTTAAAGAAAGCT |
| | | GACAAAATATTAATTTTGCATGAAGGTAGCAGCTATTTTTATG |
| | | GGACATTTTCAGAACTCCAAAATCTACAGCCAGACTTTAGCT |
| | | CAAAACTCATGGGATGTGATTCTTTCGACCAATTTAGTGCAG |
| | | AAAGAAGAAATTCAATCCTAACTGAGACCTTACACCGTTTCTC |
| | | ATTAGAAGGAGATGCTCCTGTCTCCTGGACAGAAACAAAAAA |
| | | ACAATCTTTTAAACAGACTGGAGAGTTTGGGGAAAAAAGGAA |
| | | GAATTCTATTCTCAATCCAATCAACTCTACGCTTCAGGCACGA |
| | | AGGAGGCAGTCTGTCCTGAACCTGATGACACACTCAGTTAAC |
| | | CAAGGTCAGAACATTCACCGAAAGACAACAGCATCCACACG |
| | | AAAAGTGTCACTGGCCCCTCAGGCAAACTTGACTGAACTGGA |
| | | TATATATTCAAGAAGGTTATCTCAAGAAACTGGCTTGGAAATA |
| | | AGTGAAGAAATTAACGAAGAAGACTTAAAGGAGTGCCTTTTT |
| | | GATGATATGGAGAGCATACCAGCAGTGACTACATGGAACAC |
| | | ATACCTTCGATATATTACTGTCCACAAGAGCTTAATTTTTGTG |
| | | CTAATTTGGTGCTTAGTAATTTTTCTGGCAGAGGTGGCTGCT |
| | | TCTTTGGTTGTGCTGTGGCTCCTTGGAAACACTCCTCTTCAA |
| | | GACAAAGGGAATAGTACTCATAGTAGAAATAACAGCTATGCA |
| | | GTGATTATCACCAGCACCAGTTCGTATTATGTGTTTTACATTT |
| | | ACGTGGGAGTAGCCGACACTTTGCTTGCTATGGGATTCTTCA |
| | | GAGGTCTACCACTGGTGCATACTCTAATCACAGTGTCGAAAA |
| | | TTTTACACCACAAAATGTTACATTCTGTTCTTCAAGCACCTAT |
| | | GTCAACCCTCAACACGTTGAAAGCAGGTGGGATTCTTAATAG |
| | | ATTCTCCAAAGATATAGCAATTTTGGATGACCTTCTGCCTCTT |
| | | ACCATATTTGACTTCATCCAGTTGTTATTAATTGTGATTGGAG |
| | | CTATAGCAGTTGTCGCAGTTTTACAACCCTACATCTTTGTTGC |
| | | AACAGTGCCAGTGATAGTGGCTTTTATTATGTTGAGAGCATA |
| | | TTTCCTCCAAACCTCACAGCAACTCAAACAACTGGAATCTGA |
| | | AGGCAGGAGTCCAATTTTCACTCATCTTGTTACAAGCTTAAAA |
| | | GGACTATGGACACTTCGTGCCTTCGGACGGCAGCCTTACTTT |
| | | GAAACTCTGTTCCACAAAGCTCTGAATTTACATACTGCCAACT |
| | | GGTTCTTGTACCTGTCAACACTGCGCTGGTTCCAAATGAGAA |
| | | TAGAAATGATTTTTGTCATCTTCTTCATTGCTGTTACCTTCATT |
| | | TCCATTTTAACAACAGGAGAAGGAGAAGGAAGAGTTGGTATT |
| | | ATCCTGACTTTAGCCATGAATATCATGAGTACATTGCAGTGG |
| | | GCTGTAAACTCCAGCATAGATGTGGATAGCTTGATGCGATCT |
| | | GTGAGCCGAGTCTTTAAGTTCATTGACATGCCAACAGAAGGT |
| | | AAACCTACCAAGTCAACCAAACCATACAAGAATGGCCAACTC |
| | | TCGAAAGTTATGATTATTGAGAATTCACACGTGAAGAAAGAT |
| | | GACATCTGGCCCTCAGGGGGCCAAATGACTGTCAAAGATCT |
| | | CACAGCAAAATACACAGAAGGTGGAAATGCCATATTAGAGAA |
| | | CATTTCCTTCTCAATAAGTCCTGGCCAGAGGGTGGGCCTCTT |
| | | GGGAAGAACTGGATCAGGGAAGAGTACTTTGTTATCAGCTTT |
| | | TTTGAGACTACTGAACACTGAAGGAGAAATCCAGATCGATGG |
| | | TGTGTCTTGGGATTCAATAACTTTGCAACAGTGGAGGAAAGC |
| | | CTTTGGAGTGATACCACAGAAAGTATTTATTTTTTCTGGAACA |
| | | TTTAGAAAAAACTTGGATCCCTATGAACAGTGGAGTGATCAA |
| | | GAAATATGGAAAGTTGCAGATGAGGTTGGGCTCAGATCTGTG |
| | | ATAGAACAGTTTCCTGGGAAGCTTGACTTTGTCCTTGTGGAT |
| | | GGGGGCTGTGTCCTAAGCCATGGCCACAAGCAGTTGATGTG |
| | | CTTGGCTAGATCTGTTCTCAGTAAGGCGAAGATCTTGCTGCT |
| | | TGATGAACCCAGTGCTCATTTGGATCCAGTAACATACCAAAT |
| | | AATTAGAAGAACTCTAAAACAAGCATTTGCTGATTGCACAGTA |
| | | ATTCTCTGTGAACACAGGATAGAAGCAATGCTGGAATGCCAA |
| | | CAATTTTTGGTCATAGAAGAGAACAAAGTGCGGCAGTACGAT |
| | | TCCATCCAGAAACTGCTGAACGAGAGGAGCCTCTTCCGGCA |
| | | AGCCATCAGCCCCTCCGACAGGGTGAAGCTCTTTCCCCACC |
| | | GGAACTCAAGCAAGTGCAAGTCTAAGCCCCAGATTGCTGCT |
| | | CTGAAAGAGGAGACAGAAGAAGAGGTGCAAGATACAAGGCT |
| | | TTAGAGAGCAGCATAAATGTTGACATGGGACATTTGCTCATG |
| | | GAATTGGCAGGCCTAATAAAGAGCTCAGATGCATCGATCAGA |
| | | GTGTGTTGGTTTTTTGTGTGTACTGAGGAACCCCTAGTGATG |
| | | GAGTTGGCCACTCCCTCTCTGCGCGCTCGCTCGCTCACTGA |
| | | GGCCGGGCGACCAAAGGTCGCCCGACGCCCGGGCTTTGCC |
| | | CGGGCGGCCTCAGTGAGCGAGCGAGCGCGCAGAGAGGGA |
| | | GTGGCC |
| 18 | pAV- | CCACTCCCTCTCTGCGCGCTCGCTCGCTCACTGAGGCCGCC |
| | F5tg83- | CGGGCAAAGCCCGGGCGTCGGGCGACCTTTGGTCGCCCGG |
| | hCFTR- | CCTCAGTGAGCGAGCGAGCGCGCAGAGAGGGAGTGGCCAA |
| | dR (flop | CTCCATCACTAGGGGTTCCTCAGATCTGAATTCGTGGTGAGC |
| | ITR) | GTCTGGGCATGTCTGGGCATGTCTGGGCATGTCTGGGCATG |
| | vector | TCGGGCATTCTGGGCGTCTGGGCATGTCTGGGCATGTCTGG |
| | | GCATCTCGAGAACGGTGACGTGCACGCGTGGGCGGAGCCA |
| | | TCACGCAGGTTGCTATATAAGCAGAGCTCGTTTAGTGAACCG |
| | | TCAGAGTCGAGCCCGAGAGACCATGCAGAGGTCGCCTCTGG |
| | | AAAAGGCCAGCGTTGTCTCCAAACTTTTTTTCAGCTGGACCA |
| | | GACCAATTTTGAGGAAAGGATACAGACAGCGCCTGGAATTGT |
| | | CAGACATATACCAAATCCCTTCTGTTGATTCTGCTGACAATCT |
| | | ATCTGAAAAATTGGAAAGAGAATGGGATAGAGAGCTGGCTTC |
| | | AAAGAAAAATCCTAAACTCATTAATGCCCTTCGGCGATGTTTT |
| | | TTCTGGAGATTTATGTTCTATGGAATCTTTTTATATTTAGGGG |
| | | AAGTCACCAAAGCAGTACAGCCTCTCTTACTGGGAAGAATCA |
| | | TAGCTTCCTATGACCCGGATAACAAGGAGGAACGCTCTATCG |
| | | CGATTTATCTAGGCATAGGCTTATGCCTTCTCTTTATTGTGAG |
| | | GACACTGCTCCTACACCCAGCCATTTTTGGCCTTCATCACAT |
| | | TGGAATGCAGATGAGAATAGCTATGTTTAGTTTGATTTATAAG |
| | | AAGACTTTAAAGCTGTCAAGCCGTGTTCTAGATAAAATAAGTA |
| | | TTGGACAACTTGTTAGTCTCCTTTCCAACAACCTGAACAAATT |
| | | TGATGAAGGACTTGCATTGGCACATTTCGTGTGGATCGCTCC |
| | | TTTGCAAGTGGCACTCCTCATGGGGCTAATCTGGGAGTTGTT |
| | | ACAGGCGTCTGCCTTCTGTGGACTTGGTTTCCTGATAGTCCT |
| | | TGCCCTTTTTCAGGCTGGGCTAGGGAGAATGATGATGAAGTA |
| | | CAGAGATCAGAGAGCTGGGAAGATCAGTGAAAGACTTGTGA |
| | | TTACCTCAGAAATGATCGAGAACATCCAATCTGTTAAGGCAT |
| | | ACTGCTGGGAAGAAGCAATGGAAAAAATGATTGAAAACTTAA |
| | | GACAAACAGAACTGAAACTGACTCGGAAGGCAGCCTATGTG |
| | | AGATACTTCAATAGCTCAGCCTTCTTCTTCTCAGGGTTCTTTG |
| | | TGGTGTTTTTATCTGTGCTTCCCTATGCACTAATCAAAGGAAT |
| | | CATCCTCCGGAAAATATTCACCACCATCTCATTCTGCATTGTT |
| | | CTGCGCATGGCGGTCACTCGGCAATTTCCCTGGGCTGTACA |
| | | AACATGGTATGACTCTCTTGGAGCAATAAACAAAATACAGGA |
| | | TTTCTTACAAAAGCAAGAATATAAGACATTGGAATATAACTTA |
| | | ACGACTACAGAAGTAGTGATGGAGAATGTAACAGCCTTCTGG |
| | | GAGGAGGGATTTGGGGAATTATTTGAGAAAGCAAAACAAAAC |
| | | AATAACAATAGAAAAACTTCTAATGGTGATGACAGCCTCTTCT |
| | | TCAGTAATTTCTCACTTCTTGGTACTCCTGTCCTGAAAGATAT |
| | | TAATTTCAAGATAGAAAGAGGACAGTTGTTGGCGGTTGCTGG |
| | | ATCCACTGGAGCAGGCAAGACTTCACTTCTAATGATGATTAT |
| | | GGGAGAACTGGAGCCTTCAGAGGGTAAAATTAAGCACAGTG |
| | | GAAGAATTTCATTCTGTTCTCAGTTTTCCTGGATTATGCCTGG |
| | | CACCATTAAAGAAAATATCATCTTTGGTGTTTCCTATGATGAA |
| | | TATAGATACAGAAGCGTCATCAAAGCATGCCAACTAGAAGAG |
| | | GACATCTCCAAGTTTGCAGAGAAAGACAATATAGTTCTTGGA |
| | | GAAGGTGGAATCACACTGAGTGGAGGTCAACGAGCAAGAAT |
| | | TTCTTTAGCAAGAGCAGTATACAAAGATGCTGATTTGTATTTA |
| | | TTAGACTCTCCTTTTGGATACCTAGATGTTTTAACAGAAAAAG |
| | | AAATATTTGAAAGCTGTGTCTGTAAACTGATGGCTAACAAAAC |
| | | TAGGATTTTGGTCACTTCTAAAATGGAACATTTAAAGAAAGCT |
| | | GACAAAATATTAATTTTGCATGAAGGTAGCAGCTATTTTTATG |
| | | GGACATTTTCAGAACTCCAAAATCTACAGCCAGACTTTAGCT |
| | | CAAAACTCATGGGATGTGATTCTTTCGACCAATTTAGTGCAG |
| | | AAAGAAGAAATTCAATCCTAACTGAGACCTTACACCGTTTCTC |
| | | ATTAGAAGGAGATGCTCCTGTCTCCTGGACAGAAACAAAAAA |
| | | ACAATCTTTTAAACAGACTGGAGAGTTTGGGGAAAAAAGGAA |
| | | GAATTCTATTCTCAATCCAATCAACTCTACGCTTCAGGCACGA |
| | | AGGAGGCAGTCTGTCCTGAACCTGATGACACACTCAGTTAAC |
| | | CAAGGTCAGAACATTCACCGAAAGACAACAGCATCCACACG |
| | | AAAAGTGTCACTGGCCCCTCAGGCAAACTTGACTGAACTGGA |
| | | TATATATTCAAGAAGGTTATCTCAAGAAACTGGCTTGGAAATA |
| | | AGTGAAGAAATTAACGAAGAAGACTTAAAGGAGTGCCTTTTT |
| | | GATGATATGGAGAGCATACCAGCAGTGACTACATGGAACAC |
| | | ATACCTTCGATATATTACTGTCCACAAGAGCTTAATTTTTGTG |
| | | CTAATTTGGTGCTTAGTAATTTTTCTGGCAGAGGTGGCTGCT |
| | | TCTTTGGTTGTGCTGTGGCTCCTTGGAAACACTCCTCTTCAA |
| | | GACAAAGGGAATAGTACTCATAGTAGAAATAACAGCTATGCA |
| | | GTGATTATCACCAGCACCAGTTCGTATTATGTGTTTTACATTT |
| | | ACGTGGGAGTAGCCGACACTTTGCTTGCTATGGGATTCTTCA |
| | | GAGGTCTACCACTGGTGCATACTCTAATCACAGTGTCGAAAA |
| | | TTTTACACCACAAAATGTTACATTCTGTTCTTCAAGCACCTAT |
| | | GTCAACCCTCAACACGTTGAAAGCAGGTGGGATTCTTAATAG |
| | | ATTCTCCAAAGATATAGCAATTTTGGATGACCTTCTGCCTCTT |
| | | ACCATATTTGACTTCATCCAGTTGTTATTAATTGTGATTGGAG |
| | | CTATAGCAGTTGTCGCAGTTTTACAACCCTACATCTTTGTTGC |
| | | AACAGTGCCAGTGATAGTGGCTTTTATTATGTTGAGAGCATA |
| | | TTTCCTCCAAACCTCACAGCAACTCAAACAACTGGAATCTGA |
| | | AGGCAGGAGTCCAATTTTCACTCATCTTGTTACAAGCTTAAAA |
| | | GGACTATGGACACTTCGTGCCTTCGGACGGCAGCCTTACTTT |
| | | GAAACTCTGTTCCACAAAGCTCTGAATTTACATACTGCCAACT |
| | | GGTTCTTGTACCTGTCAACACTGCGCTGGTTCCAAATGAGAA |
| | | TAGAAATGATTTTTGTCATCTTCTTCATTGCTGTTACCTTCATT |
| | | TCCATTTTAACAACAGGAGAAGGAGAAGGAAGAGTTGGTATT |
| | | ATCCTGACTTTAGCCATGAATATCATGAGTACATTGCAGTGG |
| | | GCTGTAAACTCCAGCATAGATGTGGATAGCTTGATGCGATCT |
| | | GTGAGCCGAGTCTTTAAGTTCATTGACATGCCAACAGAAGGT |
| | | AAACCTACCAAGTCAACCAAACCATACAAGAATGGCCAACTC |
| | | TCGAAAGTTATGATTATTGAGAATTCACACGTGAAGAAAGAT |
| | | GACATCTGGCCCTCAGGGGGCCAAATGACTGTCAAAGATCT |
| | | CACAGCAAAATACACAGAAGGTGGAAATGCCATATTAGAGAA |
| | | CATTTCCTTCTCAATAAGTCCTGGCCAGAGGGTGGGCCTCTT |
| | | GGGAAGAACTGGATCAGGGAAGAGTACTTTGTTATCAGCTTT |
| | | TTTGAGACTACTGAACACTGAAGGAGAAATCCAGATCGATGG |
| | | TGTGTCTTGGGATTCAATAACTTTGCAACAGTGGAGGAAAGC |
| | | CTTTGGAGTGATACCACAGAAAGTATTTATTTTTTCTGGAACA |
| | | TTTAGAAAAAACTTGGATCCCTATGAACAGTGGAGTGATCAA |
| | | GAAATATGGAAAGTTGCAGATGAGGTTGGGCTCAGATCTGTG |
| | | ATAGAACAGTTTCCTGGGAAGCTTGACTTTGTCCTTGTGGAT |
| | | GGGGGCTGTGTCCTAAGCCATGGCCACAAGCAGTTGATGTG |
| | | CTTGGCTAGATCTGTTCTCAGTAAGGCGAAGATCTTGCTGCT |
| | | TGATGAACCCAGTGCTCATTTGGATCCAGTAACATACCAAAT |
| | | AATTAGAAGAACTCTAAAACAAGCATTTGCTGATTGCACAGTA |
| | | ATTCTCTGTGAACACAGGATAGAAGCAATGCTGGAATGCCAA |
| | | CAATTTTTGGTCATAGAAGAGAACAAAGTGCGGCAGTACGAT |
| | | TCCATCCAGAAACTGCTGAACGAGAGGAGCCTCTTCCGGCA |
| | | AGCCATCAGCCCCTCCGACAGGGTGAAGCTCTTTCCCCACC |
| | | GGAACTCAAGCAAGTGCAAGTCTAAGCCCCAGATTGCTGCT |
| | | CTGAAAGAGGAGACAGAAGAAGAGGTGCAAGATACAAGGCT |
| | | TTAGAGAGCAGCATAAATGTTGACATGGGACATTTGCTCATG |
| | | GAATTGGCAGGCCTAATAAAGAGCTCAGATGCATCGATCAGA |
| | | GTGTGTTGGTTTTTTGTGTGTACTGAGGAACCCCTAGTGATG |
| | | GAGTTGGCCACTCCCTCTCTGCGCGCTCGCTCGCTCACTGA |
| | | GGCCGGGCGACCAAAGGTCGCCCGACGCCCGGGCTTTGCC |
| | | CGGGCGGCCTCAGTGAGCGAGCGAGCGCGCAGAGAGGGA |
| | | GTGGCCCCCCCCCCCCCCCCCCCTGCAGCCTGGCGTAATA |
| | | GCGAAGAGGCCCGCACCGATCGCCCTTCCCAACAGTTGCGC |
| | | AGCCTGAATGGCGAATGGACGCGCCCTGTAGCGGCGCATTA |
| | | AGCGCGGCGGGTGTGGTGGTTACGCGCAGCGTGACCGCTA |
| | | CACTTGCCAGCGCCCTAGCGCCCGCTCCTTTCGCTTTCTTCC |
| | | CTTCCTTTCTCGCCACGTTCGCCGGCTTTCCCCGTCAAGCTC |
| | | TAAATCGGGGGCTCCCTTTAGGGTTCCGATTTAGTGCTTTAC |
| | | GGCACCTCGACCCCAAAAAACTTGATTAGGGTGATGGTTCAC |
| | | GTAGTGGGCCATCGCCCTGATAGACGGTTTTTCGCCCTTTGA |
| | | CGTTGGAGTCCACGTTCTTTAATAGTGGACTCTTGTTCCAAA |
| | | CTGGAACAACACTCAACCCTATCTCGGTCTATTCTTTTGATTT |
| | | ATAAGGGATTTTGCCGATTTCGGCCTATTGGTTAAAAAATGA |
| | | GCTGATTTAACAAAAATTTAACGCGAATTTTAACAAAATATTA |
| | | ACGCTTACAATTTCCTGATGCGGTATTTTCTCCTTACGCATCT |
| | | GTGCGGTATTTCACACCGCATATGGTGCACTCTCAGTACAAT |
| | | CTGCTCTGATGCCGCATAGTTAAGCCAGCCCCGACACCCGC |
| | | CAACACCCGCTGACGCGCCCTGACGGGCTTGTCTGCTCCCG |
| | | GCATCCGCTTACAGACAAGCTGTGACCGTCTCCGGGAGCTG |
| | | CATGTGTCAGAGGTTTTCACCGTCATCACCGAAACGCGCGA |
| | | GACGAAAGGGCCTCGTGATACGCCTATTTTTATAGGTTAATG |
| | | TCATGATAATAATGGTTTCTTAGACGTCAGGTGGCACTTTTCG |
| | | GGGAAATGTGCGCGGAACCCCTATTTGTTTATTTTTCTAAATA |
| | | CATTCAAATATGTATCCGCTCATGAGACAATAACCCTGATAAA |
| | | TGCTTCAATAATATTGAAAAAGGAAGAGTATGAGTATTCAACA |
| | | TTTCCGTGTCGCCCTTATTCCCTTTTTTGCGGCATTTTGCCTT |
| | | CCTGTTTTTGCTCACCCAGAAACGCTGGTGAAAGTAAAAGAT |
| | | GCTGAAGATCAGTTGGGTGCACGAGTGGGTTACATCGAACT |
| | | GGATCTCAACAGCGGTAAGATCCTTGAGAGTTTTCGCCCCGA |
| | | AGAACGTTTTCCAATGATGAGCACTTTTAAAGTTCTGCTATGT |
| | | GGCGCGGTATTATCCCGTATTGACGCCGGGCAAGAGCAACT |
| | | CGGTCGCCGCATACACTATTCTCAGAATGACTTGGTTGAGTA |
| | | CTCACCAGTCACAGAAAAGCATCTTACGGATGGCATGACAGT |
| | | AAGAGAATTATGCAGTGCTGCCATAACCATGAGTGATAACAC |
| | | TGCGGCCAACTTACTTCTGACAACGATCGGAGGACCGAAGG |
| | | AGCTAACCGCTTTTTTGCACAACATGGGGGATCATGTAACTC |
| | | GCCTTGATCGTTGGGAACCGGAGCTGAATGAAGCCATACCA |
| | | AACGACGAGCGTGACACCACGATGCCTGTAGCAATGGCAAC |
| | | AACGTTGCGCAAACTATTAACTGGCGAACTACTTACTCTAGC |
| | | TTCCCGGCAACAATTAATAGACTGGATGGAGGCGGATAAAGT |
| | | TGCAGGACCACTTCTGCGCTCGGCCCTTCCGGCTGGCTGGT |
| | | TTATTGCTGATAAATCTGGAGCCGGTGAGCGTGGGTCTCGC |
| | | GGTATCATTGCAGCACTGGGGCCAGATGGTAAGCCCTCCCG |
| | | TATCGTAGTTATCTACACGACGGGGAGTCAGGCAACTATGGA |
| | | TGAACGAAATAGACAGATCGCTGAGATAGGTGCCTCACTGAT |
| | | TAAGCATTGGTAACTGTCAGACCAAGTTTACTCATATATACTT |
| | | TAGATTGATTTAAAACTTCATTTTTAATTTAAAAGGATCTAGGT |
| | | GAAGATCCTTTTTGATAATCTCATGACCAAAATCCCTTAACGT |
| | | GAGTTTTCGTTCCACTGAGCGTCAGACCCCGTAGAAAAGATC |
| | | AAAGGATCTTCTTGAGATCCTTTTTTTCTGCGCGTAATCTGCT |
| | | GCTTGCAAACAAAAAAACCACCGCTACCAGCGGTGGTTTGTT |
| | | TGCCGGATCAAGAGCTACCAACTCTTTTTCCGAAGGTAACTG |
| | | GCTTCAGCAGAGCGCAGATACCAAATACTGTTCTTCTAGTGT |
| | | AGCCGTAGTTAGGCCACCACTTCAAGAACTCTGTAGCACCG |
| | | CCTACATACCTCGCTCTGCTAATCCTGTTACCAGTGGCTGCT |
| | | GCCAGTGGCGATAAGTCGTGTCTTACCGGGTTGGACTCAAG |
| | | ACGATAGTTACCGGATAAGGCGCAGCGGTCGGGCTGAACGG |
| | | GGGGTTCGTGCACACAGCCCAGCTTGGAGCGAACGACCTAC |
| | | ACCGAACTGAGATACCTACAGCGTGAGCATTGAGAAAGCGC |
| | | CACGCTTCCCGAAGGGAGAAAGGCGGACAGGTATCCGGTAA |
| | | GCGGCAGGGTCGGAACAGGAGAGCGCACGAGGGAGCTTCC |
| | | AGGGGGAAACGCCTGGTATCTTTATAGTCCTGTCGGGTTTCG |
| | | CCACCTCTGACTTGAGCGTCGATTTTTGTGATGCTCGTCAGG |
| | | GGGGCGGAGCCTATGGAAAAACGCCAGCAACGCGGCCTTTT |
| | | TACGGTTCCTGGCCTTTTGCTGGCCTTTTGCTCACATGTTCTT |
| | | TCCTGCGTTATCCCCTGATTCTGTGGATAACCGTATTACCGC |
| | | CTTTGAGTGAGCTGATACCGCTCGCCGCAGCCGAACGACCG |
| | | AGCGCAGCGAGTCAGTGAGCGAGGAAGCGGAAGAGCGCCC |
| | | AATACGCAAACCGCCTCTCCCCGCGCGTTGGCCGATTCATTA |
| | | ATGCAGGCTGCAGGGGGGGGGGGGGGGGGG |

### Example 2: Repeat Dosing of AV.TL65 to Ferret Lungs Elicits an Antibody Response that Diminishes Transduction in an Age-Dependent Manner

Repeat-dosing of recombinant adeno-associated virus (rAAV) may be necessary to treat cystic fibrosis (CF) lung disease using gene therapy. However, little is known about rAAV-mediated immune responses in the lung. Here we demonstrate that the ferret is a suitable species for the preclinical testing of AV.TL65 for CFTR delivery to the lung and characterization of neutralizing antibody (NAb) responses. AV.TL65-hCFTRΔR efficiently transduced both human and ferret airway epithelial cultures, and complemented CFTR Cl⁻ currents in CF airway cultures. Delivery of AV.TL65-hCFTRΔR to neonatal and juvenile ferret lungs produced hCFTR mRNA at 200-300% greater levels than endogenous fCFTR. Single-dose (AV.TL65-gLuc) or repeat-dosing (AV.TL65-fCFTRΔR followed by AV.TL65-gLuc) of AV.TL65 was performed in neonatal and juvenile ferrets. Repeat-dosing significantly reduced transgene expression (11-fold) and increased bronchioalveolar lavage fluid (BALF) NAbs in juvenile but not neonatal ferrets, despite near equivalent plasma NAbs responses in both age groups. Notably, both age groups demonstrated a reduction in BALF anti-capsid binding IgG, IgM, and IgA antibodies following repeat-dosing. Unique to juvenile ferrets was a suppression of plasma anti-capsid binding IgM following the second vector administration. Thus, age-dependent immune system maturation and isotype switching may impact the development of high-affinity lung NAbs following repeat-dosing of AV.TL65 and may provide a path to blunt AAV neutralizing responses in the lung.

The above results were carried out as follows in greater detail below.

Results

The Ferret is a Suitable Preclinical Species for Evaluation of AV.TL65 Gene Therapy to the Lung

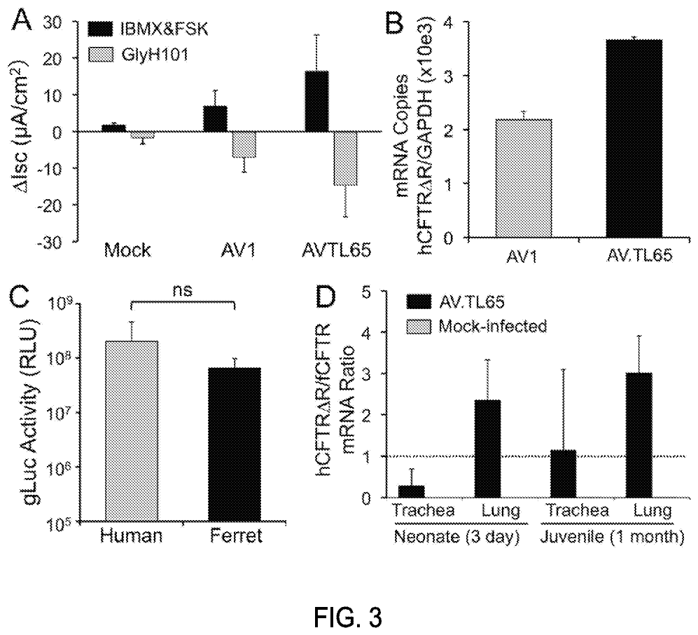

To evaluate whether the AV.TL65 (AV2.5T) capsid variant was capable of complementing CFTR function in the airway, we tested the ability of AV.TL65-SP183-hCFTRΔR virus to correct CFTR-mediated Cl⁻ current in human CF ALI cultures following apical infection. Because rAAV1 had been previously shown to be one of the best performing serotypes for apically transduction of human ALI cultures, we also pseudopackaged the same AV2-F5tg83-hCFTRΔR viral genome into the AAV1 capsid and performed a comparative analysis with AV.TL65. This comparison demonstrated that apical infection with AV.TL65-SP183-hCFTRΔR virus gave rise to higher levels of CFTR-mediated Cl⁻ current (FIG. 3A) and CFTR mRNA (FIG. 3B) than that following infection with the rAAV1 virus harboring the same genome (AV1.SP183-hCFTRΔR).

To evaluate whether AV.TL65 was also capable to transducing ferret airway epithelium, we first performed in vitro transduction assays in well-differentiated tracheobronchial ALI cultures derived from humans and ferrets using a secreted Gaussia luciferase (gLuc) reporter vector, AV.TL65-SP183gLuc (FIG. 3C). Apical infection of these cultures with AV.TL65-SP183gLuc demonstrated no significant difference in the levels of gLuc transgene expression between the two species. To confirm the tropism of AV.TL65 for ferret lungs in vivo, we evaluated the transduction efficiency of AV.TL65-SP183-hCFTRΔR in neonatal and juvenile ferret following intratracheal delivery. In these studies, expression of the transgene-derived hCFTRΔR mRNA was referenced to endogenous fCFTR mRNA as an index (i.e., the ratio of hCFTRΔR/fCFTR mRNA copies) for the efficiency of transduction. Using this metric, hCFTRΔR mRNA expression in the lungs was 2- to 3-fold greater than endogenous fCFTR mRNA in both neonates and juvenile ferrets (FIG. 3D). By contrast, tracheal expression of hCFTRΔR mRNA was lower than endogenous fCFTR mRNA in neonates and near equivalent in juvenile animals. The low neonatal and highly variable juvenile transduction of the trachea with AV.TL65 was potentially due to the delivery method, which used surgery to instill the virus into the middle of the trachea. Overall, these in vitro and in vivo studies indicate that the ferret is a suitable species to study immunologic responses in the lung to AV.TL65 infection.

Previous Exposure of AV.TL65 to Lungs of Juvenile, but not Neonatal, Ferrets Impairs Transduction by a Second Administration

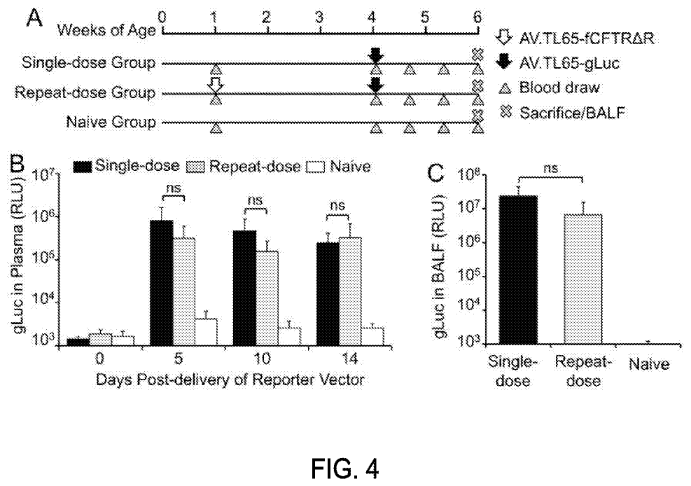

We utilized two rAAV vectors (AV.TL65-SP183-fCFTRΔR and AV.TL65-SP183-gLuc) to evaluate the feasibility of repeat-dosing of AV.TL65 to the ferret lung. AV.TL65-SP183-fCFTRΔR was chosen for the first viral infection, since this vector should not mount an immune response to the transgene (i.e., ferret CFTR or fCFTR). For the second viral infection, we wanted a robust reporter that would allow for temporal and quantitative analysis of transgene expression and thus chose a secreted gLuc reporter vector, AV.TL65-SP183-gLuc. The ferrets in the single-dose groups were infected with only the AV.TL65-SP183-gLuc vector and those of the repeat-dose group were infected first with AV.TL65-SP183-fCFTRΔR and second with AV.TL65-SP183-gLuc. We first evaluated the repeated dosing in younger animals (FIG. 4). We initiated these studies in neonatal ferrets, infecting the repeat-dose group at 1 week of age with AV.TL65-SP183-fCFTRΔR and then three weeks later infecting both the repeat-dose and single-dose (naive) groups with AV.TL65-SP183-gLuc virus (FIG. 4A). Luciferase activity was monitored in blood samples during the 14 days post-infection with AV.TL65-SP183-gLuc and in BALF at the termination of the experiment. Finding from this study demonstrated that gLuc activity in plasma peaked by 5-days post-infection and remained stable to 14 days in both dosing groups (FIG. 4B). There was also no significant difference in the level of plasma gLuc activity between the two dosing groups. Similarly, gLuc activity in the BALF at 14 days post-infection was also not significantly different between the two dosing groups (FIG. 4C). In both the plasma and BALF, gLuc activity was well above background levels in naive (uninfected) controls (FIGS. 4B and 4C).

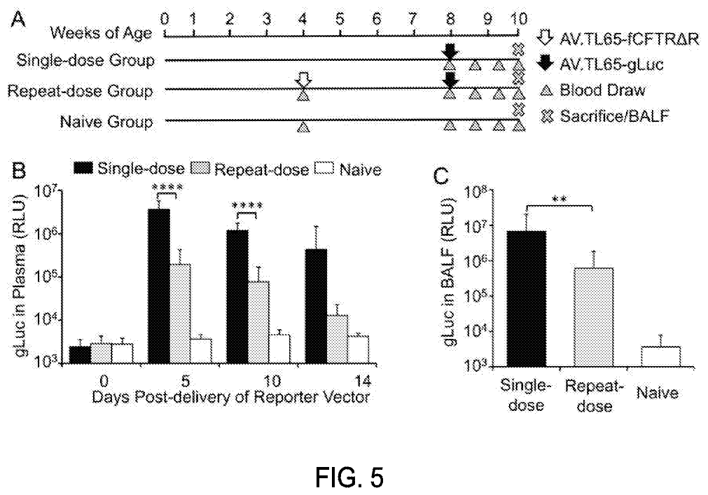

This study in neonatal ferrets demonstrated it was feasible to re-administer AV.TL65 without a significant decline in transduction to the lung; however, the possibility remained that an underdeveloped immune system in neonatal ferrets could produce a tolerized immunologic state against the AAV capsid. For these reasons, we repeated experiments in juvenile ferrets by initiating the first infection with AV.TL65-SP183-fCFTRΔR for the repeat-dose group at 1 month of age, which approximately represents a 1-2 years old toddler, followed the delivery of the gLuc reporter vector (AV.TL65-SP183-gLuc) to both the single-dose and repeat-dose groups 4 weeks later (FIG. 5A). Findings from this second study demonstrated maximal plasma gLuc activity at 5-days post-infection in both groups, however, the repeat-dose group had lower (15- to 34-fold) plasma gLuc activity at all time points tested. In contrast to the stable plasma gLuc expression in single- and repeated-dose neonatal groups (FIG. 4B), we observed a gradually declined in plasma gLuc activity in both juvenile groups with steeper trend in the repeat-dose animals. (FIG. 5B). Similarly, BALF gLuc activity was also significantly lower (11-fold) in the repeat-dose juvenile group (FIG. 5C). Cumulatively, these studies suggested the potential for NAb responses against the AAV capsid in juvenile but not neonatal ferrets.

Repeat-Dosing of AV.TL65 Elicits a Higher NAb Response in the BALF and Plasma

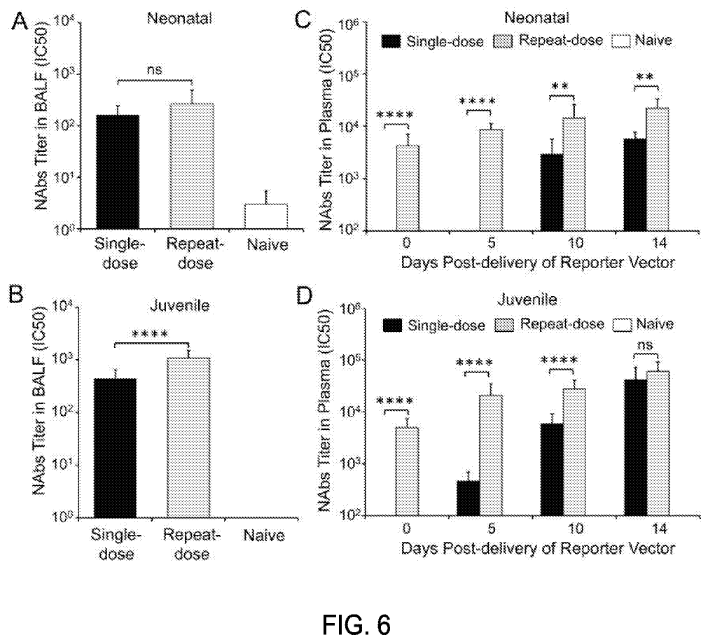

Given the reduced efficiency of AV.TL65 transduction in the lungs of juvenile ferrets previously exposed to this virus, we sought to evaluate the NAbs in the BALF and plasma of test animals. The titers of anti-AV.TL65 NAbs were determined as the IC₅₀ for inhibition of AV.TL65-SP183-fLuc transduction in A594 cells, an human airway cell line. Consistent with similar levels of transgene expression in single- and repeat-dosed neonatal ferret, NAb titers in BALF were not significantly different between the two dosing conditions (FIG. 6A). By contrast, NAb titers in the BALF of juvenile ferrets were significantly higher in the repeat-dose as compared to the single-dose group (FIG. 6B). Furthermore, the absolute titers of NAbs in experiments with older animals of both single and repeat dose groups were higher (3- to 5-fold) than the neonatal test groups, suggestive of a more fully developed immune response in the older ferrets.

Similar analyses on the plasma samples demonstrated no pre-existing NAbs in the control naive group (FIGS. 6C and D) and the test groups prior to AV.TL65 infection. In both age groups, single- and repeat-dose animals demonstrated gradual time-dependent increases in plasma NAb titers following infection and repeat-dose juvenile ferrets produced slightly higher plasma NAb titers (2-2.8 fold) than did neonatal ferrets. Juvenile ferrets also produced NAbs more rapidly in the plasma following single-dose infection with an appearance at 5-days post-infection as compared to 10-days for neonatal ferrets. The level of plasma NAbs in the repeat-dose group was also significantly higher than that of single-dose groups for both ages, with the exception of the 14-days post-infection time point in the juvenile ferrets.

Development of an ELISA-Based Assay for Quantifying Anti-AV.TL65 Capsid Antibody Isotypes

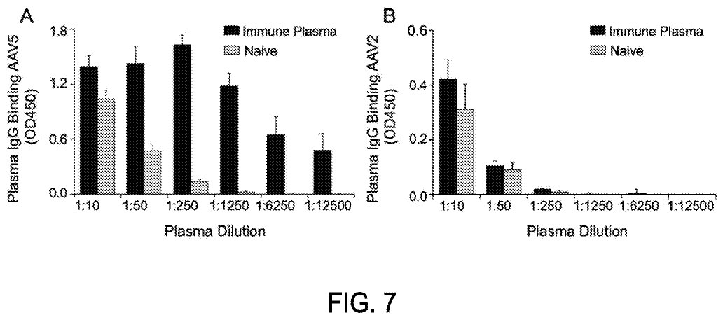

Evolved from an AAV2/AAV5 capsid-shuffling library, VP2 and the most abundant VP3 capsid proteins of AV.TL65 are derived from AAV5 with a single A581T mutation in VP1. VP1 of AV.TL65 is a hybrid of AAV2 and AAV5 capsids with the N-terminal unique sequence (VP1u) from the 1-131 aa of the AAV2 VP1 following by 128-724 aa of AAV5 capsid harboring the A581T mutation. The VP1u of AAV harbors a phospholipase A2 (PLA2) catalytic domain that is thought to be crucial to virion escape from the endosome. To evaluate AV.TL65 capsid-specific immunoglobins in the plasma and BALF (IgG, IgM, and IgA) of AV.TL65-infected ferrets, an ELISA assay using AAV viral particles as the coating antigen was developed. To validate the method, we used plasma collected from a 1-month-old ferret for which AV.TL65 virus was delivered to the lung four times at 1-2 months intervals. Using AAV5 particles as the coating antigen, differential IgG binding between naive and AV.TL65-immune plasma was seen starting at a 1:50 dilution, and by a 1:1250 dilution binding of naive plasma was absent while AV.TL65-immune plasma antibody binding remained high (FIG. 7A). By contrast, when AAV2 was used as the coating antigens, there was no difference in plasma IgG binding between the immune plasma and the naive plasma at all dilutions and the sensitivity of detecting IgG was much lower than AAV5 (FIG. 7B). These findings suggest the surface antigen epitopes of AV.TL65 displays similar immunogenicity to the AAV5 capsid and for these reasons we chose to use AAV5 as the coating antigen for classification of anti-capsid antibody isotypes in the BALF and plasma of test animals.

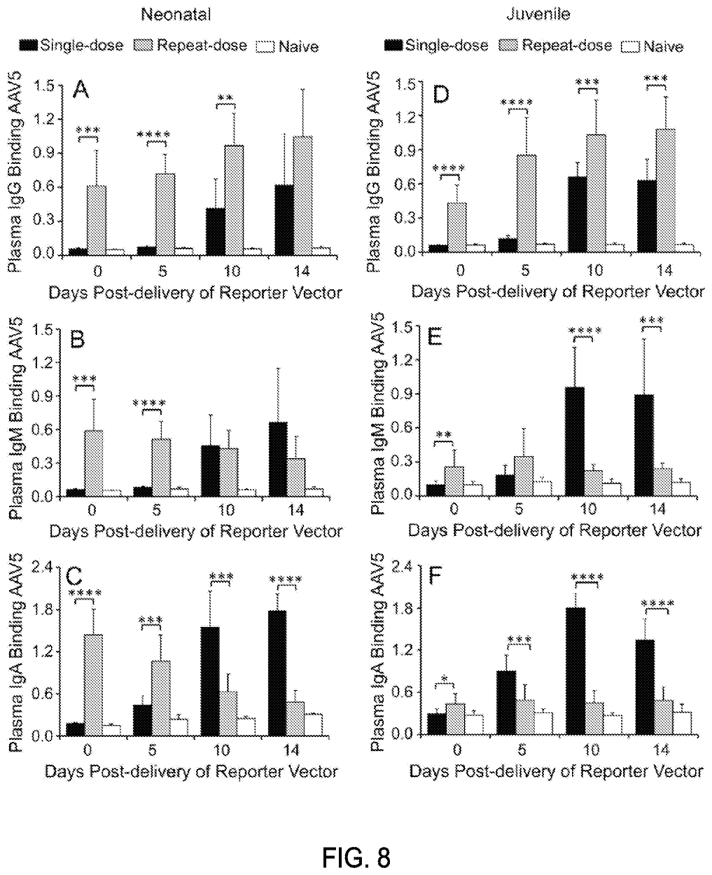

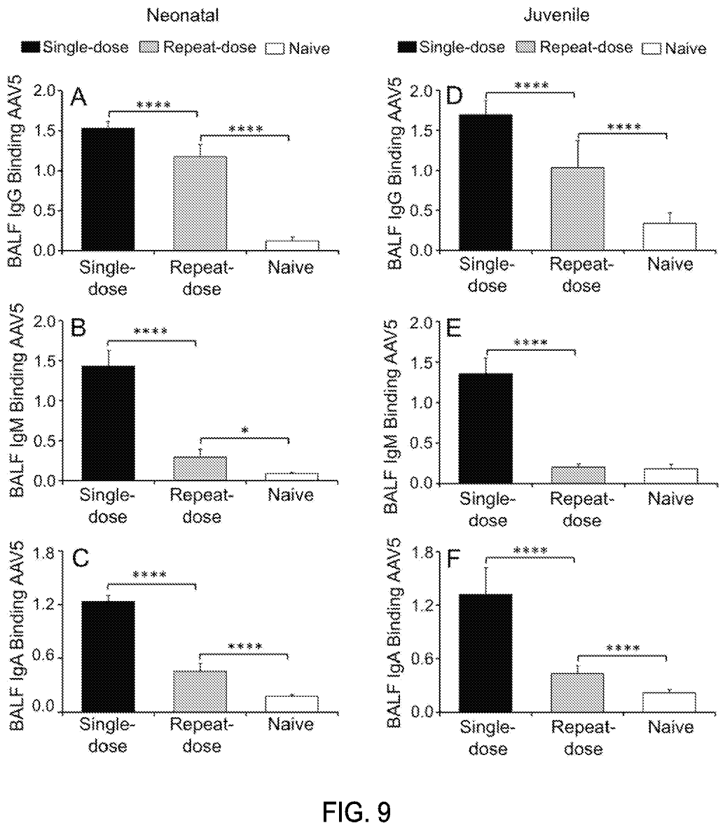

We next used this ELISA method for classification of anti-capsid antibody isotypes (IgG, IgM, and IgA) in the BALF and plasma of test animals (FIGS. 7 and 8). In general, neonatal and juvenile ferrets elicited similar AAV5-reactive IgG responses in the plasma of both single- and repeat-dosing groups, but titers were higher following repeat-infection (FIGS. 8A and 8D). By contrast, plasma AAV5-reactive IgM (FIGS. 8B and 8E) and IgA (FIGS. 8C and F) responses demonstrated differences from that of IgG with respect to age of the animal and dosing regimen. For example, capsid-binding plasma IgM levels were suppressed only in juvenile animals of the repeat-dose group (FIGS. 8B and 8E), while capsid-binding plasma IgA levels were suppressed in both age groups following repeat dosing. Furthermore, neonatal animals initially mounted a large anti-capsid IgA response initially following second viral expose which subsided with time, while juvenile animals lacked this response (FIGS. 8C and 8F). These findings suggest that age-dependent differences in antibody isotype switching may be impacted by prior expose to AV.TL65. Contrary to expectations, AAV5-reactive IgG, IgM and IgA in the BALF was significantly higher in the single-dose group, as compared to the repeat-dose group, for both neonatal and juvenile animals (FIG. 9). Furthermore, the absolute level of capsid-binding IgG, IgM and IgA were generally similar between both age groups and dosing conditions, despite higher levels of NAbs in the BALF of juvenile animals that were exposed twice to virus (FIGS. 6A and 6B).

Materials and Methods

Production of Recombinant AV.TL65 Viral Vectors

pAV.TL65repcap (Excoffon et al., 2009, supra) was the AAV helper plasmid used to generate AV.TL65 capsid for the production of AV1-SP183-hCFTRΔR, and AV.TL65-SP183-hCFTRΔR, AV.TL65-SP183-fCFTRΔR, AV.TL65-SP183-fLuc, AV.TL65-SP183-gLuc. rAAV proviral plasmids used for packaging were pAV2.F5tg83-hCFTRΔR and pAV2.F5tg83-fCFTRΔR, as well as the pAV2-F5tg83fLuc (firefly luciferase reporter) and pAV2-F5tg83gLuc (Gaussia luciferase reporter). AV.TL65 vectors were produced in the Vector Core of Children's Hospital of Philadelphia (CHOP) using a triple-plasmid transfection method. In brief, AAV helper pAV.TL65repcap and Adenovirus helper pAd were transfected into HEK293 cells together with one of the AAV proviral vector. rAAV vector produced from the transfected HEK293 cells were purified on CsCl-density gradients. The titers were determined by quantitative real-time polymerase chain reaction (qPCR) using primers and probes specific to the transgenes, and the purity of the vector stocks were evaluated by SDS-PAGE following silver-staining.

In Vitro Evaluation of AV.TL65 Vector in Human and Ferret Airway Epithelium

In order to evaluate whether the ferret would be a suitable species for analysis of AV.TL65, we initially performed in vitro transduction experiments in well-differentiated tracheobronchial ALI cultures derived from humans and ferrets. The reporter vector, AV.TL65-SP183gLuc, was inoculated apically onto the airway epithelial ALI cultures of human (n=6 transwells from two donors) and ferret (n=6 transwells from two donors) at an MOI (multiplicity of infection) of 10,000 DRP (DNase-resistant particle)/cell. During the infection period, the culture medium was supplemented with doxorubicin at the final concentration of 4 μM, and the relative luminescence units (RLU) of Gaussia luciferase activity was measured after 5-days infection according to the manufacturer's instructions for the Renilla Luciferase activity assay kit (Promega), which was designed for the measurement of Gaussia luciferase and Renilla luciferase. Two non-infected transwells were set as control.

In Vitro Comparison of CFTR-Mediated Currents Following Infection of Human CF Airway Epithelium with AV1-SP183-hCFTRΔR and AV.TL65-SP183-hCFTRΔR Viruses

The effectiveness of AV.TL65-SP183-hCFTRΔR and AV1-SP183-hCFTRΔR for expressing hCFTRΔR and complementation of CFTR function was evaluated in polarized human ALI cultures derived from the proximal airway of CF patients (F508del/F508del). Each vector was apically applied to the ALI cultures (n=4 transwells from two donors) at an MOI of 100,000 DRP/cell in the presence of doxorubicin (2.5 μM) and LLnL (20 μM). These two proteasome modulating agents have been shown to augment transduction by several AAV serotypes. At 12-day post-infection, CFTR-mediated CI-currents were measured in Ussing chambers as described previously to determine the change in short-circuit current (ΔIsc) following cAMP stimulation (IBMX/Forskolin) and CFTR inhibition (GlyH101). Non-infected ALI cultures (n=4 transwells from two donors) were used as baseline controls. After measure of the ΔIsc, two inserts from each virus infection group were pooled and lysed for total RNA using the RNeasy® Plus Mini kit (Qiagene). After conversion of mRNA to cDNA, the vector-derived hCFTRΔR mRNA was quantitated by TaqMan® PCR and normalized to human GAPDH mRNA.

Analysis of AV.TL65 Transduction in Neonatal and Juvenile Ferret Lungs

Three-day-old neonatal ferrets (n=3) or one-month-old juvenile ferrets (n=3) intratracheally received 4×10¹⁰ DRP per gram body weight of the AV.TL65-SP183-hCFTRΔR virus mixing with doxorubicin (final concentration 250 μM). The ferrets in the mocked infection group (n=3) were only inoculated with Dox in PBS (250 μM). The animals were euthanized at 11-days post-infection, the trachea and lung tissues were separately harvested, snap frozen, and pulverized for total RNA extraction. The vector-derived mRNA of the transgene hCFTRΔR and endogenous fCFTR were quantified by TaqMan®, and the copy numbers of hCFTRΔR and fCFTRΔR were normalized to GAPDH and then expressed as the ratio of hCFTRΔR/fCFTR.

Administration of AV.TL65-SP183-fCFTRΔR and/or AV.TL65-SP183-gLuc to Ferrets for Humoral Response Studies

We evaluated repeat dosing of AV.TL65 vectors to neonatal and juvenile ferrets using the following experimental design. Neonatal ferrets: AV.TL65-SP183-gLuc reporter vector was intratracheally administered to 4-week-old ferrets that were either naive to AV.TL65 capsid or previously infected with AV.TL65-SP183-fCFTΔR at 1-week of age. Juvenile ferrets: AV.TL65-SP183-gLuc reporter vector was intratracheally administered to 8-week-old ferrets that were either naive to AV.TL65 capsid or previously infected with AV.TL65-SP183-fCFTRΔR at 4-weeks of age. For each dose, the animal received an inoculum containing AV.TL65-SP183gLuc or AV.TL65-SP183-fCFTRΔR vector (1×10¹³ DRP/kg) and doxorubicin (200 μM final concentration). Surgical intratracheal injection was performed in the 1-week-old neonatal ferrets with a 150 μl inoculum administered to kits under anesthesia with a mixture of isofluorane and oxygen. For other ages, virus was administered intratracheally with a MicroSprayer® aerosolizer under anesthesia via subcutaneous injection with a mixture of ketamine and xylazine. The volume of the vector/doxorubicin inoculum for aerosolization was normalized to ferret body weight (5 ml/kg).

Bleeding and Bronchoalveolar Lavage Fluid Collection for Measurement of Gaussia Luciferase Activity

Plasma was collected into heparinized tubes from anesthetized ferrets at the 0-, 5-, 10- and 14-days post-delivery of the AV.TL65-SP183-gLuc report vector. Animals were euthanized with EUTHASOL® (Virbac AH Inc) and bronchoalveolar lavage fluid (BALF) was collected from the tracheal/lung cassette by instillation of 5 ml of PBS per 300-gram body weight. The gLuc activity in plasma and BALF were immediately measured after sample collection.

Antibody Neutralization Assays Using Plasma and BALF

Micro-neutralization assays were performed using modifications to a previously reported method (Wu et al. Front Immunol. 8:1649, 2017). The titer of NAb in the plasma and BALF was quantified as the reduction in reporter gene expression following infection of A549 cells with AV.TL65-SP183-fLuc virus incubated with serially diluted plasma or BALF prior to infection. Briefly, all plasma samples from ferrets were heat-inactivated (56° C., 30 min). Five-fold serial dilutions of plasma (started at 1:50 and ended at 1:156,250) were incubated with AV.TL65-SP183-fLuc in a total volume of 100 μl. For BALF, the same condition was applied, but the serial dilution started at 1:5 and ended at 1:3125. These mixtures were incubated at 37° C. for 1 hr to facilitate antibody binding and neutralization, and then applied to a monolayer of A549 cells in 48-well plates (1×10⁵/well, MOI=5000 DRP/cell) in duplicate for each dilution. After incubating cells for 1 hr at 37° C./5% CO₂ with the virus mixture, the wells supplemented with DMEM containing of 2% fetal bovine serum and incubated for an additional 24 hrs. Firefly Luciferase activity in cell lysates were then measured with a Firefly Luciferase Assay Kit (Promega) according to manufacturer's instruction. Each time this assay was performed, A549 cells infected only with AV.TL65-SP183-fLuc served as the reference control for 100% transduction. The neutralization titer of each plasma or BALF sample was calculated as the half maximal inhibitory concentration (IC50).

ELISA Measurements of Capsid-Binding IgG, IgM, and IgA in Plasma and BALF

An ELISA procedure was used to capture and quantify the total capsid-binding IgG, IgM, and IgA in the plasma and BALF. In brief, rAAV5 in carbonate buffer was bound to 96 wells ELISA plates overnight at 4° C. (1×10⁹ DRP/well). The tested plasma samples (diluted to 1:2000 for IgG and IgM and 1:20 for IgA) and undiluted BALF samples were applied to each well, and incubated for 1 hr at room temperature. After washing three times in PBS-T (0.05% Tween-20), diluted HRP-conjugated second antibodies were added and incubated for 1 hr at room temperature. The HRP-conjugated second antibodies included chicken anti-ferret IgG (Gallus Immunotech or Abcam) and goat anti-ferret IgM or IgA (Life-Bio Inc). The HRP reaction product was then quantified by absorbance in a plate reader.

Statistical Analysis

Experimental data are presented as mean±SD and Prism 7 (GraphPad Software, Inc., San Diego, Calif., USA) was used for data analysis. The statistical significance was analyzed with one-way analysis of variance (ANOVA) followed by Tukey test (*P<0.05; **P<0.01; ***P<0.001, ****P<0.0001).

Ethics Statement in Animal Care

All animal experimentation was performed according to protocols approved by the Institutional Animal Care and Use Committees of the University of Iowa.

All publications, patents and patent applications are incorporated herein by reference. While in the foregoing specification, this invention has been described in relation to certain some embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method of expressing a transgene in a cell, the method comprising contacting the cell with (i) a recombinant adeno-associated virus (rAAV) comprising an AV.TL65 capsid protein, or a variant thereof, and a polynucleotide comprising a transgene; and (ii) an augmenter of AAV transduction, thereby expressing the transgene in the cell.

2. The method of claim 1, wherein the augmenter is a proteasome modulating agent.

3. The method of claim 2, wherein the proteasome modulating agent is an anthracycline, a proteasome inhibitor, a tripeptidyl aldehyde, or a combination thereof.

4. The method of claim 3, wherein the anthracycline comprises doxorubicin, idarubicin, aclarubicin, daunorubicin, epirubicin, valrubicin, mitoxantrone, or a combination thereof.

5. (canceled)

6. The method of claim 3, wherein the proteasome inhibitor comprises bortezomib, carfilzomib, or ixazomib.

7. The method of claim 3, wherein the tripeptidyl aldehyde is N-acetyl-l-leucyl-l-leucyl-l-norleucine (LLnL).

8. The method of claim 1, wherein the cell is contacted sequentially with the rAAV and the augmenter.

9. The method of claim 1, wherein the cell is contacted simultaneously with the rAAV and the augmenter.

10. The method of claim 1, wherein contacting the cell with the rAAV and the augmenter results in an increase in expression of the transgene as compared to contacting the cell with the rAAV alone.

11. (canceled)

12. The method of claim 1, wherein the contacting comprises administering the rAAV and the augmenter to a subject.

13. A method of treating a disorder in a subject in need thereof, the method comprising administering to the subject (i) a recombinant adeno-associated virus (rAAV) comprising an AV.TL65 capsid protein, or a variant thereof, and a polynucleotide comprising a therapeutic transgene; and (ii) an augmenter of AAV transduction, wherein the administering results in expression of the transgene in cells of the subject.

14. The method of claim 12, wherein the administering is by inhalation, by nebulization, or by aerosolization, or is intranasal, intratracheal, intrabronchial, oral, intravenous, subcutaneous, and/or intramuscular administration.

15. (canceled)

16. The method of claim 1, wherein the cell is an airway epithelial cell.

17. (canceled)

18. The method of claim 13, wherein the disorder is cystic fibrosis.

19. The method of claim 1, wherein the transgene is CFTR or a CFTRΔR derivative thereof.

20. (canceled)

21. The method of claim 13, wherein the AV.TL65 capsid protein comprises the amino acid sequence of
(SEQ ID NO: 13)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPFGLVEEGAKTAPTGKRIDDHFPKRK
KARTEEDSKPSTSSDAEAGPSGSQQLQIPAQPASSLGADTMSAGGGGPLGD
NNQGADGVGNASGDWHCDSTWMGDRVVTKSTRTWVLPSYNNHQYREIKSGS
VDGSNANAYFGYSTPWGYFDFNRFHSHWSPRDWQRLINNYWGFRPRSLRVK
IFNIQVKEVTVQDSTTTIANNLTSTVQVFTDDDYQLPYVVGNGTEGCLPAF
PPQVFTLPQYGYATLNRDNTENPTERSSFFCLEYFPSKMLRTGNNFEFTYN
FEEVPFHSSFAPSQNLFKLANPLVDQYLYRFVSTNNTGGVQFNKNLAGRYA
NTYKNWFPGPMGRTQGWNLGSGVNRASVSAFATTNRMELEGASYQVPPQPN
GMTNNLQGSNTYALENTMIFNSQPANPGTTATYLEGNMLITSESETQPVNR
VAYNVGGQMATNNQSSTTAPTTGTYNLQEIVPGSVWMERDVYLQGPIWAKI
PETGAHFHPSPAMGGFGLKHPPPMMLIKNTPVPGNITSFSDVPVSSFITQY
STGQVTVEMEWELKKENSKRWNPEIQYTNNYNDPQFVDFAPDSTGEYRTTR
PIGTRYLTRPL.

22. A pharmaceutical composition comprising (i) an rAAV comprising an AV.TL65 capsid protein, or a variant thereof, and a polynucleotide comprising a transgene; and (ii) an augmenter of AAV transduction.

23. The pharmaceutical composition of claim 22, wherein the augmenter is a proteasome modulating agent.

24. The pharmaceutical composition of claim 23, wherein the proteasome modulating agent is an anthracycline, a proteasome inhibitor, a tripeptidyl aldehyde, or a combination thereof.

25. The pharmaceutical composition of claim 24, wherein the anthracycline comprises doxorubicin, idarubicin, aclarubicin, daunorubicin, epirubicin, valrubicin, mitoxantrone, or a combination thereof.

26-28. (canceled)
